(19) United States
(12) Patent Application Publication
(10) Pub. No.: US 2024/0279336 A1
(43) Pub. Date: Aug. 22, 2024

(54) CHIMERIC PROTEINS AND METHODS OF IMMUNOTHERAPY

(71) Applicants: INSERM (INSTITUT NATIONAL DE LA SANTÉ ET DE LA RECHERCHE MÉDICALE), Paris (FR); NANTES UNIVERSITÉ, Nantes (FR)

(72) Inventors: Nicolas BOISGERAULT, Nantes (FR); Tacien PETITHOMME, Nantes (FR)

(21) Appl. No.: 18/291,271

(22) PCT Filed: Jul. 29, 2022
(86) PCT No.: PCT/EP2022/071407

§ 371 Date: Jan. 23, 2024

(30) Foreign Application Priority Data
Jul. 30, 2021 (EP) 21306069.2

(51) Int. Cl. C07K 16/28; A61K 38/00; A61P 35/00; C07K 14/705; C07K 16/30; C12N 15/86

(52) U.S. Cl. CPC C07K 16/2827; A61P 35/00; C07K 14/70575; C07K 16/2818; C07K 16/30; C12N 15/86; A61K 38/00; C07K 2317/569; C07K 2317/622; C07K 2319/02; C07K 2319/30; C07K 2319/33; C12N 2760/20243

(57) ABSTRACT

Chimeric proteins for cancer immunotherapy can combine different activities by displaying both agonistic and antagonistic properties on a single molecule. However, those usually exhibit toxicities related to their potency being exerted in the entire organism and not only in tissues relevant for cancer treatment. This may be solved by using appropriate vectorization of the chimeric proteins to the tumour microenvironment, where immune cells interact with tumour cells expressing immunomodulatory molecules. The present invention relates to a nucleic acid encoding a chimeric protein or a chimeric protein having a general structure of: N terminus-(a)-(b)-(c)-C terminus, wherein: (a) is a signaling and/or targeting domain. (b) is a linker (b) is a functional linker and (c) is a signaling and/or targeting domain or N terminus-(c)-(b)-(a)-C terminus, wherein: (c) is a signaling and/or targeting domain. (b) is a linker (b) is a functional linker and (a) is a signaling and/or targeting domain.

### FIELD OF THE INVENTION:

The invention is in the field of immunotherapy. More particularly, the invention relates to methods and compositions for selecting a cancer treatment in a subject suffering from cancer.

### BACKGROUND OF THE INVENTION:

A fusion protein is an artificial protein obtained by the combination of different proteins, or parts of proteins. It is obtained following the creation by recombination of DNA of a gene comprising the open reading frames corresponding to the desired proteins or parts of proteins. Fusion proteins can also be called chimeric proteins.

Classical therapeutic proteins for cancer immunotherapy (e.g. antibodies, ligands) target immunomodulatory proteins located on the surface of both tumour cells and immune cells for either agonistic or antagonistic activity. Chimeric proteins for cancer immunotherapy can combine these activities by displaying both agonistic and antagonistic properties on a single molecule. However, those usually exhibit toxicities related to their potency being exerted in the entire organism and not only in tissues relevant for cancer treatment. This may be solved by using appropriate vectorization of the chimeric proteins to the tumour microenvironment, where immune cells interact with tumour cells expressing immunomodulatory molecules. This vectorization can be achieved by inserting the genes coding for the chimeric proteins in gene therapy vectors, including viral vectors such as oncolytic viruses that specifically replicate in tumours. This specific vectorization to the tumours guarantees onsite expression of the potent chimeric proteins, which is expected to improve their anti-tumour efficacy while limiting their toxicity. This enhanced safety offers opportunities for further engineering of the chimeric proteins that can be supplemented with additional functional domains, which will activate other immune mechanisms and thus reinforce their immunostimulatory properties.

### SUMMARY OF THE INVENTION:

The present invention relates to methods and compositions for selecting a cancer treatment in a subject suffering from cancer. In particular, the present invention is defined by the claims.

### DETAILED DESCRIPTION OF THE INVENTION

The inventors built novel immunotherapeutic molecules to specifically and precisely reverse cell-targeted immune responses in different pathological contexts (e.g. cancer, transplantation, allergy, autoimmune diseases).

In the context of cancer immunotherapy—in which the aim is to enhance the immune responses directed against the targeted tumor cells—these molecules will improve the immune recognition of the targeted cells and the activation of the neighboring immune cells to elicit specific, localized and long-lasting anti-tumor immune responses.

In the contexts of autoimmune diseases, allergies, and transplantation-for which the aim is to minimize the immune responses against the targeted self or transplanted cells-these molecules will inhibit the immune recognition of the targeted cells and tolerize the neighboring immune cells to protect the patient from cellular and tissular destruction over time.

### The Chimeric Protein

In a first object, the present injection relates to a nucleic acid encoding a chimeric protein having a general structure of:
N terminus-(a)-(b)-(c)-C terminus, wherein: (a) is a signaling and/or targeting domain, (b) is a functional linker and (c) is a signaling and/or targeting domain or
N terminus-(c)-(b)-(a)-C terminus, wherein: (c) is a signaling and/or targeting domain, (b) is a functional linker and (a) is a signaling and/or targeting domain.

In some embodiments, the present invention relates to a nucleic acid encoding a chimeric protein having a general structure of: N terminus-(a)-(b)-(c)-C terminus wherein (a) is a domain, (b) is a functional linker and (c) is a domain.

In some embodiments, the present invention relates to a nucleic acid encoding a chimeric protein having a general structure of: N terminus-(c)-(b)-(a)-C terminus wherein (a) is a domain, (b) is a functional linker and (c) is a domain.

In some embodiments, the present invention relates to a nucleic acid encoding a chimeric protein having a general structure of: N terminus-(a)-(b)-(c)-C terminus wherein (a) is a signaling and/or targeting domain, (b) is a functional linker and (c) is a signaling and/or targeting domain.

In some embodiments, the present invention relates to a nucleic acid encoding a chimeric protein having a general structure of: N terminus-(c)-(b)-(a)-C terminus wherein (a) is a signaling and/or targeting domain, (b) is a functional linker and (c) is a signaling and/or targeting domain.

In some embodiments, the present invention relates to a nucleic acid encoding a chimeric protein having a general structure of: N terminus-(a)-(b)-(c)-C terminus wherein (a) is a monomeric domain, (b) is a functional linker and (c) is a trimeric (e.g. homotrimeric or heterotrimeric) domain.

In some embodiments, the present invention relates to a nucleic acid encoding a chimeric protein having a general structure of: N terminus-(c)-(b)-(a)-C terminus wherein (c) is a monomeric domain, (b) is a functional linker and (a) is a trimeric (e.g. homotrimeric or heterotrimeric) domain.

In some embodiments, the present invention relates to a nucleic acid encoding a chimeric protein having a general structure of: N terminus-(a)-(b)-(c)-C terminus wherein (a) is a monomeric domain, (b) is a functional linker and (c) is a dimeric (e.g. homodimeric, heterodimeric) domain.

In some embodiments, the present invention relates to a nucleic acid encoding a chimeric protein having a general structure of: N terminus-(c)-(b)-(a)-C terminus wherein (c) is a monomeric domain, (b) is a functional linker and (a) is a dimeric (e.g. homodimeric, heterodimeric) domain.

In a second object, the present injection relates to a chimeric protein having a general structure of:
N terminus-(a)-(b)-(c)-C terminus, wherein: (a) is a signaling and/or targeting domain, (b) is a functional linker and (c) is a signaling and/or targeting domain or
N terminus-(c)-(b)-(a)-C terminus, wherein: (c) is a signaling and/or targeting domain, (b) is a functional linker and (a) is a signaling and/or targeting domain.

In some embodiments, the present invention relates to a chimeric protein having a general structure of: N terminus-(a)-(b)-(c)-C terminus wherein (a) is a domain, (b) is a functional linker and (c) is a domain.

In some embodiments, the present invention relates to a chimeric protein having a general structure of: N terminus-(c)-(b)-(a)-C terminus wherein (a) is a domain, (b) is a functional linker and (c) is a domain.

In some embodiments, the present invention relates to a chimeric protein having a general structure of: N terminus-(a)-(b)-(c)-C terminus wherein (a) is a signaling and/or targeting domain, (b) is a functional linker and (c) is a signaling and/or targeting domain.

In some embodiments, the present invention relates to a chimeric protein having a general structure of: N terminus-(c)-(b)-(a)-C terminus wherein (a) is a signaling and/or targeting domain, (b) is a functional linker and (c) is a signaling and/or targeting domain.

In some embodiments, the present invention relates to a chimeric protein having a general structure of: N terminus-(a)-(b)-(c)-C terminus wherein (a) is a targeting monomeric domain, (b) is a functional linker and (c) is a trimeric (e.g. homotrimeric or heterotrimeric) domain.

In some embodiments, the present invention relates to a chimeric protein having a general structure of: N terminus-(c)-(b)-(a)-C terminus wherein (c) is a monomeric domain, (b) is a functional linker and (a) is a trimeric (e.g. homotrimeric or heterotrimeric) domain.

In some embodiments, the present invention relates to a chimeric protein having a general structure of: N terminus-(a)-(b)-(c)-C terminus wherein (a) is a monomeric domain, (b) is a functional linker and (c) is a dimeric (e.g. homodimeric, heterodimeric) domain.

In some embodiments, the present invention relates to a chimeric protein having a general structure of: N terminus-(c)-(b)-(a)-C terminus wherein (c) is a monomeric domain, (b) is a functional linker and (a) is a dimeric (e.g. homodimeric, heterodimeric) domain.

In some embodiments, the chimeric protein of the invention is composed of a targeting domain (a) and/or a targeting domain (c).

In some embodiments, the present invention relates to a chimeric protein having a general structure of: N terminus-(a)-(c)-(b)-C terminus wherein (c) is a monomeric domain, (b) is a functional linker and (a) is a dimeric (e.g. homodimeric, heterodimeric) domain or a trimeric (e.g. homotrimeric or heterotrimeric) domain.

In some embodiments, the chimeric protein of the invention has a targeting domain (a) that specifically binds to molecules at the surface of the cells to be targeted for immune destruction or protection or immune reprogramming. In some embodiments, the chimeric protein has a targeting domain (c) that specifically binds to molecules at the surface of the cells to be targeted for immune destruction or protection or immune reprogramming.

In some embodiments, the chimeric protein of the invention is composed of a signaling domain (a) and/or a signaling domain (c).

In some embodiments, the chimeric protein of the invention has a signaling domain (a) that initiates a signal transduction cascade. In some embodiments, the chimeric protein has a signaling domain (c) that initiates a signal transduction cascade.

In some embodiments, the chimeric protein of the invention has a domain (a) which is a targeting domain and/or a signaling domain. In some embodiments, the chimeric protein of the invention has a domain (c) which is a targeting domain and/or a signaling domain.

As used herein, the term “chimeric protein” refers to proteins created through the joining of two or more genes that originally coded for separate proteins. Translation of this fusion gene results in a single or multiple polypeptides with functional properties derived from each of the original proteins. The chimeric protein comprises a first amino acid sequence linked to a second amino acid sequence with which it is not naturally linked in nature. The amino acid sequences may normally exist in separate proteins that are brought together in the fusion protein or they may normally exist in the same protein but are placed in a new arrangement in the fusion protein. A chimeric protein may be created, for example, by chemical synthesis, or by creating and translating a polynucleotide in which the peptide regions are encoded in the desired relationship.

As used herein the term “TAME-IT” refers to the chimeric protein of the present invention.

In some embodiments, the chimeric protein of the invention is capable of binding murine ligand(s)/receptor(s). In some embodiments, the chimeric protein of the invention is capable of binding human ligand(s)/receptor(s).

In some embodiments, the chimeric protein is an activator protein.

In some embodiments, the chimeric protein is engineered to enhance, increase, and/or stimulate the transmission of an immune stimulatory signal.

In some embodiments, the chimeric protein is an inhibitor protein.

In some embodiments, the chimeric protein is engineered to inhibit, decrease, and/or block the transmission of an immune stimulatory signal.

In some embodiments, the chimeric protein of the invention comprises an anti-PD-L1 and is paired with a monomeric or dimeric (e.g. homodimeric, heterodimeric) or trimeric (e.g. homotrimeric or heterotrimeric) immune stimulatory agent as follows: anti-PD-L1/monomeric or dimeric (e.g. homodimeric, heterodimeric) or trimeric (e.g. homotrimeric or heterotrimeric) 4-1 BBL; anti-PD-L1/monomeric or dimeric (e.g. homodimeric, heterodimeric) or trimeric (e.g. homotrimeric or heterotrimeric) OX-40L; anti-PD-L1/monomeric or dimeric (e.g. homodimeric, heterodimeric) or trimeric (e.g. homotrimeric or heterotrimeric) LIGHT; anti-PD-L1/monomeric or dimeric (e.g. homodimeric, heterodimeric) or trimeric (e.g. homotrimeric or heterotrimeric) GITRL; anti-PD-L1/monomeric or dimeric (e.g. homodimeric, heterodimeric) or trimeric (e.g. homotrimeric or heterotrimeric) CD70; anti-PD-L1/monomeric or dimeric (e.g. homodimeric, heterodimeric) or trimeric(e.g. homotrimeric or heterotrimeric) CD30L; anti-PD-L1/monomeric or dimeric (e.g. homodimeric, heterodimeric) or trimeric (e.g. homotrimeric or heterotrimeric) CD40L; anti-PD-L1/monomeric or dimeric (e.g. homodimeric, heterodimeric) or trimeric (e.g. homotrimeric or heterotrimeric) HVEM, anti-PD-L1/monomeric or dimeric or trimeric (e.g. homotrimeric or heterotrimeric) GITR; anti-PD-L1/monomeric or dimeric (e.g. homodimeric, heterodimeric) or trimeric (e.g. homotrimeric or heterotrimeric) CD27; anti-PD-L1/monomeric or dimeric (e.g. homodimeric, heterodimeric) or trimeric (e.g. homotrimeric or heterotrimeric) CD28, anti-PD-L1/monomeric or dimeric (e.g. homodimeric, heterodimeric) or trimeric (e.g. homotrimeric or heterotrimeric) CD137 and/or anti-PD-L1/monomeric or dimeric (e.g. homodimeric, heterodimeric) or trimeric (e.g. homotrimeric or heterotrimeric) TL1A. In some embodiments the chimeric protein is anti-PD-L1-Fc-LIGHT monomeric or dimeric (e.g. homodimeric, heterodimeric) or trimeric (e.g. homotrimeric or heterotrimeric) or anti-PD-L1-Fc-OX40L monomeric or dimeric (e.g. homodimeric, heterodimeric) or trimeric (e.g. homotrimeric or heterotrimeric), in which the Fc represents a linker that comprises at least a portion of an Fc domain of an antibody and which comprises at least one cysteine residue capable of forming a disulfide bond. In some embodiments, the anti-PD-L1 is a nanobody or a scFv. In a particular embodiment, the anti-PD-L1 nanobody is KN035 (see Zhang et al. Cell Discov. 2017; 3: 17004) or 5DXW (see https://www.rcsb.org/structure/5DXW). In a particular embodiment, the anti-PD-L1 scFv is aPD-L1.

In some embodiments, the chimeric protein comprises anti-PD-L2 and is paired with a monomeric or dimeric (e.g. homodimeric, heterodimeric) or trimeric (e.g. homotrimeric or heterotrimeric) immune stimulatory receptor as follows: anti-PD-L2/monomeric or dimeric (e.g. homodimeric, heterodimeric) or trimeric (e.g. homotrimeric or heterotrimeric) 4-1 BBL; anti-PD-L2/monomeric or dimeric (e.g. homodimeric, heterodimeric) or trimeric (e.g. homotrimeric or heterotrimeric) OX-40; anti-PD-L2/monomeric or dimeric (e.g. homodimeric, heterodimeric) or trimeric (e.g. homotrimeric or heterotrimeric) HVEM; anti-PD-L2/monomeric or dimeric (e.g. homodimeric, heterodimeric) or trimeric (e.g. homotrimeric or heterotrimeric) GITR; anti-PD-L2/monomeric or dimeric (e.g. homodimeric, heterodimeric) or trimeric (e.g. homotrimeric or heterotrimeric) CD27; anti-PD-L2/monomeric or dimeric (e.g. homodimeric, heterodimeric) or trimeric (e.g. homotrimeric or heterotrimeric) CD28; anti-PD-L2/monomeric or dimeric (e.g. homodimeric, heterodimeric) or trimeric (e.g. homotrimeric or heterotrimeric) CD30; anti-PD-L2/monomeric or dimeric (e.g. homodimeric, heterodimeric) or trimeric (e.g. homotrimeric or heterotrimeric) CD40 and anti-PD-L2/monomeric or dimeric (e.g. homodimeric, heterodimeric) or trimeric (e.g. homotrimeric or heterotrimeric) CD137.

In some embodiments, the chimeric protein of the invention comprises an anti-TIM-3 and is paired with a monomeric or dimeric (e.g. homodimeric, heterodimeric) or trimeric (e.g. homotrimeric or heterotrimeric) immune stimulatory agent as follows: anti-TIM-3/monomeric or dimeric (e.g. homodimeric, heterodimeric) or trimeric (e.g. homotrimeric or heterotrimeric) OX-40L; anti-TIM-3/monomeric or dimeric (e.g. homodimeric, heterodimeric) or trimeric (e.g. homotrimeric or heterotrimeric) LIGHT; anti-TIM-3/monomeric or dimeric (e.g. homodimeric, heterodimeric) or trimeric (e.g. homotrimeric or heterotrimeric) GITRL; anti-TIM-3/monomeric or dimeric (e.g. homodimeric, heterodimeric) or trimeric (e.g. homotrimeric or heterotrimeric) CD70; anti-TIM-3/monomeric or dimeric (e.g. homodimeric, heterodimeric) or trimeric (e.g. homotrimeric or heterotrimeric) CD30L; anti-TIM-3/monomeric or dimeric (e.g. homodimeric, heterodimeric) or trimeric (e.g. homotrimeric or heterotrimeric) CD40L; anti-TIM-3/monomeric or dimeric (e.g. homodimeric, heterodimeric) or trimeric (e.g. homotrimeric or heterotrimeric) CD137L; anti-TIM-3/TL1 A monomeric or dimeric (e.g. homodimeric, heterodimeric) or trimeric (e.g. homotrimeric or heterotrimeric); and anti-TIM-3/monomeric or dimeric (e.g. homodimeric, heterodimeric) or trimeric (e.g. homotrimeric or heterotrimeric) OX40L. In embodiments the chimeric protein is anti-TIM3-Fc-OX40L monomeric or dimeric (e.g. homodimeric, heterodimeric) or trimeric (e.g. homotrimeric or heterotrimeric), in which the Fc represents a linker that comprises at least a portion of an Fc domain of an antibody and which comprises at least one cysteine residue capable of forming a disulfide bond.

In some embodiments, the chimeric protein of the invention comprises an anti-CTLA-4 and is paired with a monomeric or dimeric (e.g. homodimeric, heterodimeric) or trimeric (e.g. homotrimeric or heterotrimeric) immune stimulatory agent as follows: anti-CTLA-4/monomeric or dimeric (e.g. homodimeric, heterodimeric) or trimeric (e.g. homotrimeric or heterotrimeric) 4-1 BBL, anti-CTLA-4/monomeric or dimeric (e.g. homodimeric, heterodimeric) or trimeric (e.g. homotrimeric or heterotrimeric) OX-40L; anti-CTLA-4/monomeric or dimeric (e.g. homodimeric, heterodimeric) or (e.g. homotrimeric or heterotrimeric) trimeric LIGHT; anti-CTLA-4/monomeric or dimeric (e.g. homodimeric, heterodimeric) or (e.g. homotrimeric or heterotrimeric) trimeric GITRL; anti-CTLA-4/monomeric or dimeric (e.g. homodimeric, heterodimeric) or (e.g. homotrimeric or heterotrimeric) trimeric CD70; anti-CTLA-4/monomeric or dimeric (e.g. homodimeric, heterodimeric) or (e.g. homotrimeric or heterotrimeric) trimeric CD30L; anti-CTLA-4/monomeric or dimeric (e.g. homodimeric, heterodimeric) or t(e.g. homotrimeric or heterotrimeric) rimeric CD40L; anti-CTLA-4/monomeric or dimeric (e.g. homodimeric, heterodimeric) or (e.g. homotrimeric or heterotrimeric) trimeric CD137L; anti-CTLA-4/monomeric or dimeric (e.g. homodimeric, heterodimeric) or (e.g. homotrimeric or heterotrimeric) trimeric TL1A; and anti-CTLA-4/monomeric or dimeric (e.g. homodimeric, heterodimeric) or trimeric (e.g. homotrimeric or heterotrimeric) OX40L. In some embodiments, the anti-CTLA-4 is a scFv. In a particular embodiment, the anti-CTLA-4 scFv is aCTLA-4 (see Griffin et al. J Immunol 2000 May 1;164(9):4433-42).

In some embodiments, the chimeric protein of the invention comprises an anti-TIGIT and is paired with a monomeric or dimeric (e.g. homodimeric, heterodimeric) or trimeric immune stimulatory agent as follows: anti-TIGIT/monomeric or dimeric (e.g. homodimeric, heterodimeric) or trimeric (e.g. homotrimeric or heterotrimeric) 4-1 BBL, anti-TIGIT/monomeric or dimeric (e.g. homodimeric, heterodimeric) or trimeric (e.g. homotrimeric or heterotrimeric) OX-40L; anti-TIGIT/monomeric or dimeric (e.g. homodimeric, heterodimeric) or trimeric (e.g. homotrimeric or heterotrimeric) LIGHT; anti-TIGIT/monomeric or dimeric (e.g. homodimeric, heterodimeric) or trimeric (e.g. homotrimeric or heterotrimeric) GITRL; anti-TIGIT/monomeric or dimeric (e.g. homodimeric, heterodimeric) or trimeric (e.g. homotrimeric or heterotrimeric) CD70; anti-TIGIT/monomeric or dimeric (e.g. homodimeric, heterodimeric) or trimeric (e.g. homotrimeric or heterotrimeric) CD30L; anti-TIGIT/monomeric or dimeric (e.g. homodimeric, heterodimeric) or trimeric (e.g. homotrimeric or heterotrimeric) CD40L; anti-TIGIT/monomeric or dimeric (e.g. homodimeric, heterodimeric) or trimeric (e.g. homotrimeric or heterotrimeric) CD137L; anti-TIGIT/monomeric or dimeric (e.g. homodimeric, heterodimeric) or trimeric (e.g. homotrimeric or heterotrimeric) TL1A; and anti-TIGIT/monomeric or dimeric or trimeric (e.g. homotrimeric or heterotrimeric) OX40L.

In some embodiments, the chimeric protein of the invention comprises an anti-VISTA and is paired with a monomeric or dimeric (e.g. homodimeric, heterodimeric) or trimeric (e.g. homotrimeric or heterotrimeric) immune stimulatory agent as follows: anti-VISTA/4-1 BBL monomeric or dimeric (e.g. homodimeric, heterodimeric) or trimeric (e.g. homotrimeric or heterotrimeric), anti-VISTA/OX-40L monomeric or dimeric (e.g. homodimeric, heterodimeric) or trimeric (e.g. homotrimeric or heterotrimeric); anti-VISTA/LIGHT monomeric or dimeric (e.g. homodimeric, heterodimeric) or trimeric (e.g. homotrimeric or heterotrimeric); anti-VISTA/GITRL monomeric or dimeric (e.g. homodimeric, heterodimeric) or trimeric (e.g. homotrimeric or heterotrimeric); anti-VISTA/CD70 monomeric or dimeric (e.g. homodimeric, heterodimeric) or trimeric (e.g. homotrimeric or heterotrimeric); anti-VISTA/CD30L monomeric or dimeric (e.g. homodimeric, heterodimeric) or trimeric (e.g. homotrimeric or heterotrimeric); anti-VISTA/CD40L monomeric or dimeric (e.g. homodimeric, heterodimeric) or trimeric (e.g. homotrimeric or heterotrimeric); anti-VISTA/CD137L monomeric or dimeric (e.g. homodimeric, heterodimeric) or trimeric (e.g. homotrimeric or heterotrimeric); anti-VISTA/TL1A monomeric or dimeric (e.g. homodimeric, heterodimeric) or trimeric (e.g. homotrimeric or heterotrimeric); and anti-VISTA/OX40L monomeric or dimeric (e.g. homodimeric, heterodimeric) or trimeric (e.g. homotrimeric or heterotrimeric).

In some embodiments, the chimeric protein of the invention comprises an anti-BTLA and is paired with a monomeric or dimeric (e.g. homodimeric, heterodimeric) or trimeric immune stimulatory agent as follows: anti-BTLA/4-1 BBL monomeric or dimeric (e.g. homodimeric, heterodimeric) or trimeric (e.g. homotrimeric or heterotrimeric), anti-BTLA/OX-40L monomeric or dimeric or trimeric (e.g. homotrimeric or heterotrimeric); anti-BTLA/LIGHT monomeric or dimeric (e.g. homodimeric, heterodimeric) or trimeric (e.g. homotrimeric or heterotrimeric); anti-BTLA/GITRL trimeric (e.g. homotrimeric or heterotrimeric); anti-BTLA/CD70 monomeric or dimeric (e.g. homodimeric, heterodimeric) or trimeric (e.g. homotrimeric or heterotrimeric); anti-BTLA/CD30L monomeric or dimeric (e.g. homodimeric, heterodimeric) or trimeric (e.g. homotrimeric or heterotrimeric); anti-BTLA/CD40L monomeric or dimeric (e.g. homodimeric, heterodimeric) or trimeric (e.g. homotrimeric or heterotrimeric); anti-BTLA/CD137L monomeric or dimeric (e.g. homodimeric, heterodimeric) or trimeric (e.g. homotrimeric or heterotrimeric); anti-BTLA/TL1A monomeric or dimeric (e.g. homodimeric, heterodimeric) or trimeric (e.g. homotrimeric or heterotrimeric); and anti-BTLA/OX40L monomeric or dimeric (e.g. homodimeric, heterodimeric) or trimeric (e.g. homotrimeric or heterotrimeric).

In some embodiments, the chimeric protein of the invention comprises an anti-CD172a(SIRP1 a) and is paired with a monomeric or dimeric (e.g. homodimeric, heterodimeric) or trimeric (e.g. homotrimeric or heterotrimeric) immune stimulatory agent as follows: anti-CD172a(SIRP1 a)/monomeric or dimeric (e.g. homodimeric, heterodimeric) or trimeric (e.g. homotrimeric or heterotrimeric) 4-1 BBL, anti-CD172a(SIRP1 a)/monomeric or dimeric (e.g. homodimeric, heterodimeric) or trimeric (e.g. homotrimeric or heterotrimeric) OX-40L; anti-CD172a(SIRP1 a)/monomeric or dimeric (e.g. homodimeric, heterodimeric) or trimeric (e.g. homotrimeric or heterotrimeric) LIGHT; anti-CD172a(SIRP1 a)/monomeric or dimeric (e.g. homodimeric, heterodimeric) or trimeric (e.g. homotrimeric or heterotrimeric) CD70; anti-CD172a(SIRP1 a)/monomeric or dimeric (e.g. homodimeric, heterodimeric) or trimeric (e.g. homotrimeric or heterotrimeric) CD30L; anti-CD172a(SIRP1 a)/monomeric or dimeric or trimeric (e.g. homotrimeric or heterotrimeric) CD40L; anti-CD172a(SIRP1 a)/monomeric or dimeric (e.g. homodimeric, heterodimeric) or trimeric (e.g. homotrimeric or heterotrimeric) CD137L; anti-CD172a(SIRP1 a)/monomeric or dimeric (e.g. homodimeric, heterodimeric) or trimeric (e.g. homotrimeric or heterotrimeric) TL1A; and anti-CD172a(SIRP1 a)/monomeric or dimeric (e.g. homodimeric, heterodimeric) or trimeric (e.g. homotrimeric or heterotrimeric) OX40L. In embodiments the chimeric protein is anti-CD172a(SIRP1 a)-Fc-CD40L trimeric (e.g. homotrimeric or heterotrimeric) or anti-CD172a(SIRP1a)-Fc-LIGHT monomeric or dimeric (e.g. homodimeric, heterodimeric) or trimeric (e.g. homotrimeric or heterotrimeric), in which the Fc represents a linker that comprises at least a portion of an Fc domain of an antibody and which comprises at least one cysteine residue capable of forming a disulfide bond.

In some embodiments, the chimeric protein of the invention comprises an anti-CD115 and is paired with a monomeric or dimeric (e.g. homodimeric, heterodimeric) or trimeric (e.g. homotrimeric or heterotrimeric) immune stimulatory agent as follows: anti-CD115/monomeric or dimeric (e.g. homodimeric, heterodimeric) or trimeric (e.g. homotrimeric or heterotrimeric) OX-40L; anti-CD115/monomeric or dimeric (e.g. homodimeric, heterodimeric) or trimeric (e.g. homotrimeric or heterotrimeric) LIGHT; anti-CD115/monomeric or dimeric (e.g. homodimeric, heterodimeric) or trimeric (e.g. homotrimeric or heterotrimeric) CD70; anti-CD115/monomeric or dimeric (e.g. homodimeric, heterodimeric) or trimeric (e.g.

homotrimeric or heterotrimeric) CD30L; anti-CD1 15/monomeric or dimeric (e.g. homodimeric, heterodimeric) or trimeric (e.g. homotrimeric or heterotrimeric) CD40L; anti-CD115/monomeric or dimeric (e.g. homodimeric, heterodimeric) or trimeric (e.g. homotrimeric or heterotrimeric) CD137L; anti-CD115/monomeric or dimeric (e.g. homodimeric, heterodimeric) or trimeric (e.g. homotrimeric or heterotrimeric) TL1A; and anti-CD115/monomeric or dimeric (e.g. homodimeric, heterodimeric) or trimeric (e.g. homotrimeric or heterotrimeric) OX40L.

In some embodiments, the chimeric protein of the invention comprises an anti-TMIGD2 and is paired with a monomeric or dimeric (e.g. homodimeric, heterodimeric) or trimeric (e.g. homotrimeric or heterotrimeric) immune stimulatory agent as follows: anti-TMIGD2/monomeric or dimeric (e.g. homodimeric, heterodimeric) or trimeric (e.g. homotrimeric or heterotrimeric) OX-40L; anti-TMIGD2/monomeric or dimeric (e.g. homodimeric, heterodimeric) or trimeric (e.g. homotrimeric or heterotrimeric) LIGHT; anti-TMIGD2/monomeric or dimeric (e.g. homodimeric, heterodimeric) or trimeric (e.g. homotrimeric or heterotrimeric) GITRL; anti-TMIGD2/monomeric or dimeric (e.g. homodimeric, heterodimeric) or trimeric (e.g. homotrimeric or heterotrimeric) CD70; anti-TMIGD2/CD30L monomeric or dimeric (e.g. homodimeric, heterodimeric) or trimeric (e.g. homotrimeric or heterotrimeric); anti-TMIGD2/monomeric or dimeric (e.g. homodimeric, heterodimeric) or trimeric CD40L; anti-TMIGD2/CD137L monomeric or dimeric or trimeric (e.g. homotrimeric or heterotrimeric); anti-TMIGD2/monomeric or dimeric (e.g. homodimeric, heterodimeric) or trimeric (e.g. homotrimeric or heterotrimeric) TL1A; and antiTMIGD2/monomeric or dimeric or trimeric (e.g. homotrimeric or heterotrimeric) OX40L.

In some embodiments, the chimeric protein of the invention comprises an anti-CD200 and is paired with a monomeric or dimeric (e.g. homodimeric, heterodimeric) or trimeric (e.g. homotrimeric or heterotrimeric) immune stimulatory agent as follows: anti-CD200/monomeric or dimeric (e.g. homodimeric, heterodimeric) or trimeric (e.g. homotrimeric or heterotrimeric) OX-40L; anti-CD200/monomeric or dimeric (e.g. homodimeric, heterodimeric) or trimeric (e.g. homotrimeric or heterotrimeric) LIGHT; anti-CD200/monomeric or dimeric (e.g. homodimeric, heterodimeric) or trimeric (e.g. homotrimeric or heterotrimeric) GITRL; anti-CD200/monomeric or dimeric (e.g. homodimeric, heterodimeric) or trimeric (e.g. homotrimeric or heterotrimeric) CD70; anti-CD200/monomeric or dimeric (e.g. homodimeric, heterodimeric) or trimeric (e.g. homotrimeric or heterotrimeric) CD30L; anti-CD200/monomeric or dimeric (e.g. homodimeric, heterodimeric) or trimeric (e.g. homotrimeric or heterotrimeric) CD40L; anti-CD200/monomeric or dimeric (e.g. homodimeric, heterodimeric) or trimeric (e.g. homotrimeric or heterotrimeric) CD137L; anti-CD200/monomeric or dimeric (e.g. homodimeric, heterodimeric) or trimeric (e.g. homotrimeric or heterotrimeric) TL1A; and anti-CD200/monomeric or dimeric (e.g. homodimeric, heterodimeric) or trimeric (e.g. homotrimeric or heterotrimeric) OX40L.

In some embodiments, the chimeric protein of the invention comprises an anti-CD19 and is paired with a monomeric or dimeric (e.g. homodimeric, heterodimeric) or trimeric (e.g. homotrimeric or heterotrimeric) immune stimulatory agent as follows: anti-CD19/monomeric or dimeric (e.g. homodimeric, heterodimeric) or trimeric (e.g. homotrimeric or heterotrimeric) 4-1 BBL, anti-CD19/monomeric or dimeric (e.g. homodimeric, heterodimeric) or trimeric (e.g. homotrimeric or heterotrimeric) OX-40L; anti-CD19 monomeric or dimeric (e.g. homodimeric, heterodimeric) or trimeric (e.g. homotrimeric or heterotrimeric) LIGHT; anti-CD19/monomeric or dimeric (e.g. homodimeric, heterodimeric) or trimeric (e.g. homotrimeric or heterotrimeric) GITRL; anti-CD19 monomeric or dimeric (e.g. homodimeric, heterodimeric) or trimeric (e.g. homotrimeric or heterotrimeric) CD70; anti-CD19/monomeric or dimeric (e.g. homodimeric, heterodimeric) or trimeric (e.g. homotrimeric or heterotrimeric)

CD30L; anti-CD19/monomeric or dimeric (e.g. homodimeric, heterodimeric) or trimeric (e.g. homotrimeric or heterotrimeric) CD40L; anti-CD19/monomeric or dimeric (e.g. homodimeric, heterodimeric) or trimeric (e.g. homotrimeric or heterotrimeric) CD137L; anti-CD19/monomeric or dimeric (e.g. homodimeric, heterodimeric) or trimeric (e.g. homotrimeric or heterotrimeric) TL1A; and anti-CD19/monomeric or dimeric (e.g. homodimeric, heterodimeric) or trimeric (e.g. homotrimeric or heterotrimeric) OX40L. In some embodiments, the anti-CD19 is a nanobody or a scFv. In a particular embodiment, the anti-CD19 scFv is an aCD19 (see Ng et al. Proc Natl Acad Sci U S A. 2012 Sep. 4;109(36):14526-31).

In some embodiments, the chimeric protein of the invention comprises an anti-MSLN (i.e. anti-mesothelin) and is paired with a monomeric or dimeric (e.g. homodimeric, heterodimeric) or trimeric (e.g. homotrimeric or heterotrimeric) immune stimulatory agent as follows: anti-MSLN/monomeric or dimeric (e.g. homodimeric, heterodimeric) or trimeric (e.g. homotrimeric or heterotrimeric) 4-1 BBL, anti-MSLN/monomeric or dimeric (e.g. homodimeric, heterodimeric) or trimeric (e.g. homotrimeric or heterotrimeric) OX-40L; anti-MSLN monomeric or dimeric (e.g. homodimeric, heterodimeric) or trimeric (e.g. homotrimeric or heterotrimeric) LIGHT; anti-MSLN/monomeric or dimeric (e.g. homodimeric, heterodimeric) or trimeric (e.g. homotrimeric or heterotrimeric) GITRL; anti-MSLN monomeric or dimeric (e.g. homodimeric, heterodimeric) or trimeric (e.g. homotrimeric or heterotrimeric) CD70; anti-MSLN/monomeric or dimeric (e.g. homodimeric, heterodimeric) or trimeric (e.g. homotrimeric or heterotrimeric) CD30L; anti-MSLN/monomeric or dimeric (e.g. homodimeric, heterodimeric) or trimeric (e.g. homotrimeric or heterotrimeric) CD40L; anti-MSLN/monomeric or dimeric (e.g. homodimeric, heterodimeric) or trimeric (e.g. homotrimeric or heterotrimeric) CD137L; anti-MSLN/monomeric or dimeric (e.g. homodimeric, heterodimeric) or trimeric (e.g. homotrimeric or heterotrimeric) TL1A; and anti-MSLN/monomeric or dimeric (e.g. homodimeric, heterodimeric) or trimeric (e.g. homotrimeric or heterotrimeric) OX40L. In some embodiments, the anti-MSLN is a nanobody. In a particular embodiment, the anti-MSLN nanobody is Al (Prantner et al, J Biomed Nanotechnol. 2015 July;11(7):1201-12).

In some embodiments, the chimeric protein of the invention is a human chimeric protein. In some embodiments, the chimeric protein of the invention is a murine chimeric protein. In some embodiments, the chimeric protein of the invention is a rodent chimeric protein. In some embodiments, the chimeric protein of the invention is a feline chimeric protein a mammal. In some embodiments, the chimeric protein of the invention is canine chimeric protein. In some embodiments, the chimeric protein of the invention is a primate chimeric protein. In some embodiments, the chimeric protein of the invention is a non-human (i.e. another specie other than human) chimeric protein.

In a particular embodiment, the chimeric protein is [Anti-PD-L1]-[TAA]ₙ-[dimeric 4-1 BBL and monomeric OX-40L], wherein n is egal to 1, 2, 3, 4, 5, 6, 7, 8, 9 or 10.

In a particular embodiment, the chimeric protein is [Anti-PD-L1]-[TAA]ₙ-[dimeric OX-40L and monomeric 4-1 BBL] wherein n is egal to 1, 2, 3, 4, 5, 6, 7, 8, 9 or 10.

In a particular embodiment, the chimeric protein is [Anti-PD-L1]-[TAA]ₙ-[trimeric 4-1 BBL], wherein n is egal to 1, 2, 3, 4, 5, 6, 7, 8, 9 or 10.

In a particular embodiment, the chimeric protein is [Anti-PD-L1]-[TAA]ₙ-[trimeric OX-40L], wherein n is egal to 1, 2, 3, 4, 5, 6, 7, 8, 9 or 10.

In a particular embodiment, the chimeric protein is [Anti-MSLN]-[TAA]ₙ-[trimeric OX-40L], wherein n is egal to 1, 2, 3, 4, 5, 6, 7, 8, 9 or 10.

In a particular embodiment, the chimeric protein is [Anti-PD-L1]-[TAA]ₙ-[trimeric hCD40L], wherein n is egal to 1, 2, 3, 4, 5, 6, 7, 8, 9 or 10.

In a particular embodiment, the chimeric protein is [Anti-MSLN]-[TAA]ₙ-[trimeric hCD40L], wherein n is egal to 1, 2, 3, 4, 5, 6, 7, 8, 9 or 10.

In a particular embodiment, the chimeric protein is [Anti-MSLN]-[TAA]ₙ-[trimeric 4-1 BBL], wherein n is egal to 1, 2, 3, 4, 5, 6, 7, 8, 9 or 10.

In a particular embodiment, the chimeric protein is [Anti-CTLA-4]-[TAA]ₙ-[trimeric 4-1 BBL], wherein n is egal to 1, 2, 3, 4, 5, 6, 7, 8, 9 or 10.

In a particular embodiment, the chimeric protein is [Anti-CTLA-4]-[TAA]ₙ-[trimeric CD40L], wherein n is egal to 1, 2, 3, 4, 5, 6, 7, 8, 9 or 10.

In a particular embodiment, the chimeric protein is [Anti-CD19]-[TAA]ₙ-[trimeric CD40L], wherein n is egal to 1, 2, 3, 4, 5, 6, 7, 8, 9 or 10.

In a particular embodiment, the chimeric protein is [Anti-mPD-L1]-[TAA]ₙ-[trimeric 4-1 BBL], wherein n is egal to 1, 2, 3, 4, 5, 6, 7, 8, 9 or 10.

In a particular embodiment, the chimeric protein is [Anti-mPD-L1]-[TAA]ₙ-[dimeric 4-1 BBL], wherein n is egal to 1, 2, 3, 4, 5, 6, 7, 8, 9 or 10.

In a particular embodiment, the chimeric protein is [Anti-mPD1]-[TAA]ₙ-[trimeric 4-1 BBL], wherein n is egal to 1, 2, 3, 4, 5, 6, 7, 8, 9 or 10.

In a particular embodiment, the chimeric protein is [dimeric Anti-mPD1]-[TAA]ₙ-[trimeric 4-1 BBL], wherein n is egal to 1, 2, 3, 4, 5, 6, 7, 8, 9 or 10.

In some embodiments, the chimeric protein of the invention binds to human PD-L1 or PD-L2 with a KD of about 1 nM to about 5 nM, for example, about 1 nM, about 1.5 nM, about 2 nM, about 2.5 nM, about 3 nM, about 3.5 nM, about 4 nM, about 4.5 nM, or about 5 nM. In embodiments, the chimeric protein binds to human PD-L1 with a KD of about 5 nM to about 15 nM, for example, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 10.5 nM, about 11 nM, about 11.5 nM, about 12 nM, about 12.5 nM, about 13 nM, about 13.5 nM, about 14 nM, about 14.5 nM, or about 15 nM. In some embodiments, the chimeric protein of the invention binds to human PD-L1 or PD-L2 with a KD of about 1 nM to about 30 nM, for example about 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM; 28 nM, 29 nM, 30 nM. In some embodiments, the chimeric protein of the invention binds to human PD-L1 or PD-L2 with a KD of about 1 nM to about 1000 nM, for example about 1 nM, 100 nM, 200 nM, 300 nM, 400 nM, 500 nM, 600 nM, 700 nM, 80 nM, 900 nM, 1000 nM.

In some embodiments, the present chimeric proteins of the invention are capable of, and can be used in methods comprising, promoting immune activation (e.g., against tumors).

In some embodiments, the present chimeric proteins are capable of, and can be used in methods comprising, suppressing immune inhibition (e.g., that allows tumors to survive).

In some embodiments, the present chimeric proteins provide improved immune activation and/or improved suppression of immune inhibition due to the proximity of signaling that is provided by the chimeric nature of the constructs.

In some embodiments, the present chimeric proteins are capable of, or can be used in methods comprising, modulating the amplitude of an immune response, e.g., modulating the level of effector output.

In some embodiments, e.g., when used for the treatment of cancer, the present chimeric proteins alter the extent of immune stimulation as compared to immune inhibition to increase the amplitude of a T cell response, including, without limitation, stimulating increased levels of cytokine production, proliferation or target killing potential.

In some embodiments, the use of trimeric signaling molecules improves the therapeutic efficacy compared to monomers.

### Targeting Domain (a) or (c)

In some embodiments, the chimeric protein of the invention is composed of a targeting domain (a) and/or a targeting domain (c).

As used herein, the term “targeting domain” refers to a domain having functions of recognition and anchoring of on the surface of specific cells in tissues or organs of interest. The targeting domain of the present invention recognizes a marker on the surface of specific cells, for instance tumor cells or immune cells; this anchors and displays the TAME-IT molecule on the cell surface and may signal inside the targeted cells. The targeting domain of the present invention also can block a signal by competition.

In some embodiments, the chimeric protein of the invention is composed of a monomeric targeting domain (a) or a dimeric (e.g. homodimeric or heterodimeric) targeting domain (a) or trimeric (e.g. homotrimeric or heterotrimeric) targeting domain (a). In some embodiments, the chimeric protein of the invention is composed of a monomeric targeting domain (c) or a dimeric (e.g. homodimeric or heterodimeric) targeting domain (c) or trimeric (e.g. homotrimeric or heterotrimeric) targeting domain (c).

In some embodiment, the targeting domain (a) or the targeting domain (c) specifically binds to molecules at the surface of the cells to be targeted for immune destruction (e.g. tumor cells) or protection (e.g. transplanted cells), or immune reprogramming (e.g. dendritic cells). The targeting domain may be engineered to target one or more molecules which include but are not limited to immune modulators (e.g. PD-L1, CTLA-4, TIGIT, TIM-3, VISTA, BTLA, LAG3, CD28) and lineage-specific antigens (e.g mesothelin, CD19, HER2). The targeting domain can be made from an antibody, a derivative or any other targeting moiety (e.g. scFv, nanobody, single-domain antibody, affitin, affibody, aptamers) either in its full or a short form, or an extracellular domain of a natural cellular receptor that is known to interact with the targeted molecule (e.g. PD-1, CD80/CD86, EGF).

In some embodiments, the chimeric protein of the present invention may be engineered to target one or more molecules involved in immune inhibition, including for example: CSF1 R, CTLA-4, PD-L1 , PD-L2, PD-1 , BTLA, HVEM, TIM3, GAL9, VISTA/VSIG8, KIR, 2B4, TIGIT, CD160 (also referred to as BY55), CHK 1 and CHK2 kinases, A2aR, CEACAM (e.g., CEACAM-1, CEACAM-3 and/or CEACAM-5), and various B-7 family ligands (including, but are not limited to, B7-1, B7-2, B7-DC, B7-H1, B7-H2, B7-H3, B7-H4, B7-H5, B7-H6 and B7-H7).

In some embodiments, the chimeric protein of the present invention may be engineered to target one or more molecules including without limitation, one or more of TIM-3, BTLA, PD-1, CSF1 R, CTLA-4, CD244, CD160, TIGIT, CD172a (SIRP1 a), 2B4, VISTA, VSIG8, CD200 and TMIGD2.

In some embodiments, the chimeric protein of the present invention may be engineered to target one or more molecules that reside on human leukocytes, myeloid cell, endothelial cells including, without limitation, the extracellular domains of OX40, SLAMF4, IL-2 R a, 4-1 BB/TNFRSF9, IL-2 R β, ALCAM, BTLA, B7-1, IL-4 R, B7-H3, BLAME/SLAMFS, CEACAM1, IL-6 R, IL-7 Ra, IL-10R a, IL-10R β, IL-12 R β 1 , IL-12 R β 2, CD2, IL-13 R a 1, IL-13, CD3, CD4, ILT2/CDS5j, ILT3/CDS5k, ILT4/CDS5d, ILT5/CDS5a, lutegrin a 4/CD49d, CDS, Integrin a E/CD103, CD6, Integrin a M/CD 1 1 b, CDS, Integrin a X/CD1 1 c, Integrin β 2/CDIS, KIR/CD15S, CD27/TNFRSF7, KIR2DL1, CD2S, KIR2DL3, CD30/TNFRSFS, KIR2DL4/CD15Sd, CD31/PECAM-1, KIR2DS4, CD40 Ligand/TNFSF5, LAG-3, CD43, LAIRI, CD45, LAIR2, CDS3, Leukotriene B4-R1, CDS4/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 R γ, Osteopontin, CRACC/SLAMF7, PD-1, CRTAM, PSGL-1, CTLA-4, RANK/TNFRSF1 1A, CX3CR1 , CX3CL1, L-Selectin, SIRP β 1, SLAM, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fey RIII/CD16, TIM-6, TNFR1/TNFRSFIA, Granulysin, TNF RIII/TNFRSF1 B, TRAIL RI/TNFRSFIOA, ICAM-1/CD54, TRAIL R2/TNFRSF10B, ICAM-2/CD102, TRAILR3/TNFRSF10C, IFN-yRI, TRAILR4/TNFRSF10D, IFN-γ R2, TSLP, IL-1 R1, LIGHT, LTBR (TNFRSF3) and TSLP R.

As used herein, the term “leukocytes” also known as white blood cells (WBCs) or leucocytes, are the cells of the immune system that are involved in protecting the body against both infectious disease and foreign invaders. All leukocytes cells are produced and derived from multipotent cells in the bone marrow known as hematopoietic stem cells. Leukocytes are found throughout the body, including the blood and lymphatic system.

As used herein, the term “myeloid cell” refers to nucleated hematopoietic cells in the body, consisting of a range of cell types with diverse functions. They include monocytes, macrophages, dendritic cells and granulocytes, and make up a critical arm of the immune system.

As used herein, the term “lymphoid cell” refers to cell that play role in the immune system. There are three different lymphocyte lineages: B and T lymphocytes, small in size, and NK lymphocytes, large and granular. Lymphocytes are small white blood cells (leukocytes) found mainly in the lymph nodes and the spleen.

As used herein, the term “fibroblast” refers to a type of biological cell that synthesizes the extracellular matrix and collagen, [1] produces the structural framework (stroma) for animal tissues, and plays a critical role in wound healing. [2] Fibroblasts are the most common cells of connective tissue in animals.

As used herein, the term “endothelial cell” refers to a permeable barrier for the blood vessel and is involved in the regulation of blood flow. Endothelial cells are pivotal to applications related to wound healing, angiogenesis, inflammatory processes, blood brain barriers, diabetes and other cardiovascular diseases.

As used herein the term “Programmed death-ligand 1” (PD-L1) also known as cluster of differentiation 274 (CD274) or B7 homolog 1 (B7-H1) refers to a protein that in humans is encoded by the CD274 gene (UniProt number: Q9NZQ7). PD-L1 is a 40 kDa type 1 transmembrane protein that has been speculated to play a major role in suppressing the adaptive arm of immune system.

As used herein the term “cytotoxic T-lymphocyte-associated protein 4” (CTLA-4), also known as CD152 (cluster of differentiation 152) refers to a protein receptor that functions as an immune checkpoint and downregulates immune responses. CTLA4 is constitutively expressed in regulatory T cells but only upregulated in conventional T cells after activation (UniProt number: P16410). The CTLA-4 protein is encoded by the CTLA4 gene in humans.

As used herein the term “T cell immunoreceptor with Ig and ITIM domains” (TIGIT) refers to an immune receptor present on some T cells and natural killer cells (NK) (UniProt number: Q495A1).

As used herein the term “T-cell immunoglobulin and mucin-domain containing-3” (TIM-3) refers to a protein that in humans is encoded by the HAVCR2 gene (UniProt number: Q8TDQ0).

As used herein the term “V-domain immunoglobulin [Ig]-containing suppressor of T-cell activation” (VISTA) refers to a type I transmembrane protein that functions as an immune checkpoint and is encoded by the C10orf54 gene (UniProt number: Q9H7M9).

As used herein the term “B-and T-lymphocyte attenuator” (BTLA) refers to a protein that in humans is encoded by the BTLA gene. BTLA has also been designated as CD272 (cluster of differentiation 272) (UniProt number: Q7Z6A9).

As used herein the term “lymphocyte activation gene 3” (LAG3) refers to a protein which in humans is encoded by the LAG3 gene and is also designated CD223 (cluster of differentiation 223) (Uniprot number: P18627).

As used herein the term “Cluster of Differentiation 28” (CD28) refers to a protein expressed on T cells that provide co-stimulatory signals required for T cell activation and survival (Uniprot number: P10747).

As used herein the term “mesothelin” also known as MSLN refers to a protein that in humans is encoded by the MSLN gene. Mesothelin is a 40 kDa protein that is expressed in mesothelial cells (Uniprot number: Q13421).

As used herein the term “B-lymphocyte antigen CD19” (CD19), also known as CD19 molecule (Cluster of Differentiation 19), B-Lymphocyte Surface Antigen B4, T-Cell Surface Antigen Leu-12 and CVID3 refers to a transmembrane protein that in humans is encoded by the gene CD19. In humans, CD19 is expressed in all B lineage cells. CD19 (Uniprot number: P15391).

As used herein the term “Receptor tyrosine-protein kinase erbB-2”, also known as CD340 (cluster of differentiation 340), proto-oncogene Neu, Erbb2 (rodent), or ERBB2 (human), is a protein that in humans is encoded by the ERBB2 gene. ERBB is abbreviated from erythroblastic oncogene B, a gene isolated from avian genome. It is also frequently called HER2 (from human epidermal growth factor receptor 2) or HER2/neu.HER2″. HER2 refers to a member of the human epidermal growth factor receptor (HER/EGFR/ERBB) family. Amplification or over-expression of this oncogene has been shown to play an imp Uniprot number: P04626).

In some embodiments, the targeting domain is an antibody.

As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lambda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); IgNARs (Immunoglobulin New Antigen Receptors) of sharks, small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001 ; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. No. 6,075,181 and 6,150,584. In some embodiments, the antibody is a single domain antibody such as described in EP 0 368 684, WO 06/030220 and WO 06/003388.

In a particular embodiment, the targeting domain is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb. In some embodiment, the antibody can be any blocking antibody.

In some embodiment, the targeting domain is a single-domain antibody. As used herein the term “single domain antibody” has its general meaning in the art and refers to the single heavy chain variable domain of antibodies of the type that can be found in Camelid mammals which are naturally devoid of light chains. Such single domain antibody are also called VHH or “nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21(11):484-490; and WO 06/030220, WO 06/003388. The nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e. , camelid nanobodies are useful as reagents to detect antigens that are otherwise cryptic using classical immunological techniques, and as possible therapeutic agents. Thus yet another consequence of small size is that a nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in nanobodies being extremely thermostable, stable to extreme pH and to proteolytic digestion, and poorly antigenic. Another consequence is that nanobodies readily move from the circulatory system into tissues, and even cross the blood-brain barrier and can treat disorders that affect nervous tissue. Nanobodies can further facilitated drug transport across the blood brain barrier. See U.S. patent application 20040161738 published Aug. 19, 2004. These features combined with the low antigenicity to humans indicate great therapeutic potential. The amino acid sequence and structure of a single domain antibody can be considered to be comprised of four framework regions or “FRs” which are referred to in the art and herein as “Framework region 1” or “FR1”; as “Framework region 2” or “FR2”; as “Framework region 3 ” or “FR3”; and as “Framework region 4” or “FR4” respectively; which framework regions are interrupted by three complementary determining regions or “CDRs”, which are referred to in the art as “Complementarity Determining Region for “CDR1”; as “Complementarity Determining Region 2” or “CDR2” and as “Complementarity Determining Region 3” or “CDR3”, respectively. Accordingly, the single domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the invention, the amino acid residues of the single domain antibody are numbered according to the general numbering for VH domains given by the International ImMunoGeneTics information system aminoacid numbering (http://imgt.cines.fr/).

In some embodiments, the antigen binding fragment of the invention is grafted into non-immunoglobulin based antibodies also called antibody mimetics selected from the group consisting of an affibody, an affilin, an affitin, an adnectin, an atrimer, an evasin, a DARPin, an anticalin, an avimer, a fynomer, and a versabody.

In some embodiments, the chimeric protein of the present invention comprises an extracellular domain of a Type I membrane protein which has immune inhibitory properties.

In some embodiments, the chimeric protein is engineered to disrupt, block, reduce, and/or inhibit the transmission of an immune inhibitory signal.

In some embodiments the present chimeric proteins are capable of, or find use in methods involving, masking an inhibitory ligand on the surface of a tumor cell and replacing that immune inhibitory ligand with an immune stimulatory ligand. Accordingly, the present chimeric proteins, in embodiments are capable of, or find use in methods involving, reducing or eliminating an inhibitory immune signal and/or increasing or activating an immune stimulatory signal. For example, a tumor cell bearing an inhibitory signal (and thus evading an immune response) may be substituted for a positive signal binding on a T cell that can then attack a tumor cell. Accordingly, in some embodiments, an inhibitory immune signal is masked by the present constructs and a stimulatory immune signal is activated. Such beneficial properties are enhanced by the single construct approach of the present chimeric proteins.

In some embodiments, the chimeric protein of the present invention blocks, reduces and/or inhibits PD-1 and PD-L1 or PD-L2 and/or the binding of PD-1 with PD-L1 or PD-L2. In some embodiments, the present chimeric protein blocks, reduces and/or inhibits the activity of CTLA-4 and/or the binding of CTLA-4 with one or more of AP2M 1, CD80, CD86, SHP-2, and PPP2R5A. In some embodiments, the chimeric protein of the present invention increases and/or stimulates GITR and/or the binding of GITR with one or more of GITR ligand.

In some embodiments, the chimeric protein of the present invention increases and/or stimulates OX40 and/or the binding of OX40 with one or more of OX40 ligand.

In some embodiments, the chimeric proteins of the present invention are capable of, or find use in methods involving, enhancing, restoring, promoting and/or stimulating immune modulation.

In some embodiments, the chimeric proteins of the present invention described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g., M1 macrophages), B cells, and dendritic cells.

In some embodiments, the chimeric proteins of the present invention enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT-or PI3K-mediated signal; an anti-apoptotic signal; and/or a signal promoting and/or necessary for one or more of: proinflammatory cytokine production or T cell migration or T cell tumor infiltration.

In some embodiments, the chimeric proteins of the present invention are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment.

In some embodiments, the present chimeric proteins are capable of, or find use in methods involving, inhibiting and/or causing a decrease in recruitment of immunosuppressive cells (e.g., myeloid-derived suppressor cells (MDSCs), regulatory T cells (Tregs), tumor associated neutrophils (TANs), M2 macrophages, and tumor associated macrophages (TAMs)) to the tumor and/or tumor microenvironment (TME).

In some embodiments, the present therapies may alter the ratio of M1 versus M2 macrophages in the tumor site and/or TME to favor M1 macrophages.

In some embodiments, the chimeric proteins of the present invention are capable of, and can be used in methods comprising, inhibiting and/or reducing T cell inactivation and/or immune tolerance to a tumor, comprising administering an effective amount of a chimeric protein described herein to a subject.

In some embodiments, the chimeric proteins of the present invention are able to increase the serum levels of various cytokines including, but not limited to, one or more of IFNy, TNFa, IL-2, IL-4, IL-5, IL-6, IL-9, IL-10, IL-13, IL-17A, IL-17F, and IL-22. In some embodiments, the present chimeric proteins are capable of enhancing IL-2, IL-4, IL-5, IL-10, IL-13, IL-17A, IL-22, TNFa or IFNy in the serum of a treated subject. In some embodiments, administration of the present chimeric protein is capable of enhancing TNFa secretion.

In some embodiments, administration of the chimeric protein of the present invention is capable of enhancing superantigen mediated TNFa secretion by leukocytes. Detection of such a cytokine response may provide a method to determine the optimal dosing regimen for the indicated chimeric protein.

In some embodiments, the chimeric proteins of the present invention inhibit, block and/or reduce cell death of an anti-tumor CD8+ and/or CD4+ T cell; or stimulate, induce, and/or increase cell death of a pro-tumor T cell. T cell exhaustion is a state of T cell dysfunction characterized by progressive loss of proliferative and effector functions, culminating in clonal deletion. Accordingly, a pro-tumor T cell refers to a state of T cell dysfunction that arises during many chronic infections and cancer. This dysfunction is defined by poor proliferative and/or effector functions, sustained expression of inhibitory receptors and a transcriptional state distinct from that of functional effector or memory T cells. Exhaustion prevents optimal control of infection and tumors. In addition, an anti-tumor CD8+ and/or CD4+ T cell refers to T cells that can mount an immune response to a tumor. Illustrative pro-tumor T cells include, but are not limited to, Tregs, CD4+ and/or CD8+ T cells expressing one or more checkpoint inhibitory receptors, Th2 cells and Th17 cells. Checkpoint inhibitory receptors refers to receptors (e.g., CTLA-4, B7-H3, B7-H4, TIM-3) expressed on immune cells that prevent or inhibit uncontrolled immune responses.

In some embodiments, the present chimeric proteins are capable of, and can be used in methods comprising, increasing a ratio of effector T cells to regulatory T cells. Illustrative effector T cells include ICOS⁺ effector T cells; cytotoxic T cells {e.g., αβ TCR, CD3⁺, CD8⁺, CD45RO⁺); CD4⁺effector T cells {e.g., αβ TCR, CD3⁺, CD4⁺, CCR7⁺, CD62Lhi, IL-7R/CD127⁺); CD8⁺ effector T cells {e.g., αβ TCR, CD3⁺, CD8⁺, CCR7⁺, CD62Lhi, IL7R/CD127⁺); effector memory T cells {e.g., CD62Llow, CD44⁺, TCR, CD3⁺, IL 7R/CD127⁺, IL-15R⁺, CCR7low); central memory T cells {e.g., CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27⁺ CD62L⁻) and late effector memory T cells (CD27⁻CD62L⁻) (TemE and TemL, respectively); CD127(⁻)CD25(low/−) effector T cells; CD127( )D25( )effector T cells; CD8⁺ stem cell memory effector cells (TSCM) {e.g., CD44(low)CD62L(high)CD122(high)sca(⁺); TH1 effector T-cells {e.g., CXCR3⁺, CXCR6⁺ and CCR5⁺; or αβ TCR, CD3⁺, CD4⁺, IL-12R⁺, IFNyR⁺, CXCR3⁺), TH2 effector T cells {e.g., CCR3⁺, CCR4⁺ and CCR8⁺; or αβ TCR, CD3⁺, CD4⁺, IL-4R⁺, IL-33R⁺, CCR4⁺, IL-17RB⁺, CRTH2⁺); TH9 effector T cells {e.g., αβ TCR, CD3⁺, CD4⁺); TH17 effector T cells {e.g., αβ TCR, CD3⁺, CD4⁺, IL-23R⁺, CCR6⁺, IL-1 R⁺); CD4⁺CD45RO⁺CCR7⁺ effector T cells, CD4⁺CD45RO⁺CCR7( )effector T cells; and effector T cells secreting IL-2, IL-4 and/or IFN-γ. Illustrative regulatory T cells include ICO⁺ regulatory T cells, CD4⁺CD25⁺FOXP3⁺ regulatory T cells, CD4⁺CD25⁺ regulatory T cells, CD4⁺CD25-regulatory T cells, CD4⁺CD25high regulatory T cells, TIM-3⁺ PD-1⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺ CCR⁺ regulatory T cells, CD62L (L-selectin)⁺ regulatory T cells, CD45RBIow regulatory T cells, CD127IOW regulatory T cells, LRRC32/GARP⁺ regulatory T cells, CD39⁺ regulatory T cells, GITR⁺ regulatory T cells, LAP⁺ regulatory T cells, 1 B1 1⁺ regulatory T cells, BTLA⁺ regulatory T cells, type 1 regulatory T cells (Tr1 cells), T helper type 3 (Th3) cells, regulatory cell of natural killer T cell phenotype (NKTregs), CD8⁺ regulatory T cells, CD8⁺CD28-regulatory T cells and/or regulatory T-cells secreting IL-10, IL-35, TGF-β, TNF-a, Galectin-1 , IFN-Y and/or MCP1.

### Functional Linker

In some embodiments, the chimeric protein of the invention is composed of a linker.

In some embodiments, the functional linker (b) connects the domain (a) to the domain (c) with no particular limit of length.

In some embodiments, the functional linker (b) connects the targeting domain (a) to the signaling domain (c) with no particular limit of length.

In some embodiments, the functional linker (b) connects the signaling domain (a) to the targeting domain (c) with no particular limit of length.

In some embodiments, the functional linker (b) connects the domain (c) to the domain (a) with no particular limit of length.

In some embodiments, the functional linker (b) connects the targeting domain (c) to the signaling domain (a) with no particular limit of length.

In some embodiments, the functional linker (b) connects the signaling domain (c) to the targeting domain (a) with no particular limit of length.

In some embodiments, the functional linker (b) connects the targeting/signaling domain (a) to the signaling domain/targeting domain (c) with no particular limit of length.

In some embodiments, the functional linker (b) connects the targeting/signaling domain (c) to the signaling domain/targeting domain (a) with no particular limit of length.

In some embodiments, the chimeric protein comprises a linker. In some embodiments, the linker may be flexible, including without limitation highly flexible. In some embodiments, the linker may be rigid, including without limitation a rigid alpha helix.

As used herein, the term “linker” refers to short amino acid sequences created in nature to separate multiple domains in a single protein.

In some embodiments, the linker may be functional. For example, without limitation, the linker may function to improve the folding and/or stability, improve the expression, improve the pharmacokinetics, and/or improve the bioactivity of the present chimeric protein. In another example, the linker may function to target the chimeric protein to a particular cell type or location.

As used herein, the term “functional linker” refers to a linker having the capacity to activate immune mechanisms by itself. The functional linker of the invention contains peptide sequences of tumor antigens that will be processed and presented on major histocompatibility complex molecules by different cell types, for instance by antigen-presenting cells upon phagocytosis of TAME-IT-targeted cells or directly by targeted cells. The functional linker of the present invention induces an immune response against specific antigens (e.g. tumor-associated antigens, neoantigens, viral antigens).

As used herein, the term “Tumor-Associated Antigens” (TAA) refers to antigen molecules present on tumor cells or normal cells, including embryonic proteins, glycoprotein antigens, squamous cell antigens, etc., which have been widely used for treating a number of tumors.

As used herein, the term “antigen” refers to a molecule or molecular structure, such as may be present on the outside of a pathogen that can be bound by an antigen-specific antibody. a B-cell antigen receptor or a T-cell antigen receptor. The presence of antigens in the body normally triggers an immune response. Antigens are “targeted” by antibodies and T-cell antigen receptors. Each antibody or T-cell antigen receptor is specifically produced by the immune system to match an antigen after cells in the immune system come into contact with it; this allows a precise identification or matching of the antigen and the initiation of an adaptive response.

As used herein, the term “murine antigen” refers to a murine molecule or murine molecular structure that can be bound by an antigen-specific antibody, a B-cell antigen receptor or a T-cell antigen receptor.

As used herein, the term “virus antigen” refers to a toxin or other substance given off by a virus which causes an immune response in its host. A viral protein is an antigen specified by the viral genome that can be detected by a specific immunological response.

As used herein, the term “neoantigen” refers to mainly tumor-specific antigens generated by mutations in tumor cells, which are only expressed in tumor cells (11). Neoantigens can also be produced by viral infection, alternative splicing and gene rearrangement (12-14). They are ideal targets for T cells to recognize cancer cells and can stimulate strong anti-tumor immune response.

In some embodiments, the functional linker of the present invention is having a combination of TAA peptides. In some embodiments, the functional linker of the present invention comprises one or multiple TAA peptides. For example, the functional linker comprises at least 1, 2, 3, 4, 5, 6, 7, 8, 9, 10 TAAs peptides.

In some embodiments, the functional linker comprises the same TAA peptides.

In some embodiments, the functional linker comprises the same TAA peptide in at least 1, 2, 3, 4, 5 copies and another TAA peptide in at least 1, 2, 3, 4, 5 copies.

In some embodiments, the antigens expressed by tumors or pathogenic microorganisms like bacteria and viruses can be inserted as antigenic peptides in the linker (b) of the chimeric protein of the invention. These chimeric proteins can thus be ‘personalized’ for each patient by changing the antigenic peptides of the linker (b) using sequences from the patient's specific antigens.

In some embodiments, the linker is selected from a repeats of 4 glycines and 1 serine (G4S sequences, also known as GGGGS), a peptide tags for protein detection with a specific antibody by common laboratory techniques, one or several identified and therapeutically-relevant antigenic peptide sequences that can be presented on class I and class II molecules of the major histocompatibility complex and against which will be directed a specific immune response, for instance Tumor-Associated Antigens (TAA) (e.g. NY-ESO-1, MelanA antigen, MUC-1) or Tolerance Antigens (e.g. MBP, PLP, MOG). In a particular embodiment, the linker is TAA.

In some embodiments, the linker comprising at least one cysteine residue capable of forming a disulfide bond. As described elsewhere herein, such at least one cysteine residue capable of forming a disulfide bond is, without wishing to be bound by theory, responsible for maintain a proper multimeric state of the chimeric protein and allowing for efficient production.

In some embodiments, there is provided a method of making a stable chimeric protein comprising adjoining a Type I and Type II transmembrane protein extracellular domain with a linker comprising at least one cysteine residue capable of forming a disulfide bond such that the resultant chimeric protein is properly folded and/or forms into a stable multimeric state. In some embodiments, the linker may be derived from naturally-occurring multi-domain proteins or are empirical linkers as described, for example, in Chichili et al, (2013), Protein Sci. 22(2): 153-167, Chen et al, (2013), Adv Drug Deliv Rev. 65(10): 1357-1369, the entire contents of which are hereby incorporated by reference. In some embodiments, the linker may be designed using linker designing databases and computer programs such as those described in Chen et al., (2013), Adv Drug Deliv Rev. 65(10): 1357-1369 and Crasto et. al., (2000), Protein Eng. 13(5):309-312, the entire contents of which are hereby incorporated by reference.

In some embodiments, the linker is a synthetic linker such as (polyethylene glycol) PEG. As used herein, the term “PEG” refers to a polyether compound derived from petroleum. The structure of PEG is commonly expressed as H—(O—CH2—CH2)n—OH. PEG is intended to encompass any of the forms of PEG that have been used to derivatize other proteins, such as mono (C1-C10) alkoxy-or aryloxy-polyethylene glycol or polyethylene glycol-maleimide.

In some embodiments, the linker is a polypeptide. The terms “polypeptide” and “protein” are used interchangeably herein to refer to a polymer of amino acid residues. The terms apply to amino acid polymers in which one or more amino acid residues is an artificial chemical mimetic of a corresponding naturally occurring amino acid, as well to naturally occurring amino acids polymers and non-naturally occurring amino acid polymers. Unless otherwise indicated, a particular polypeptide sequence also implicitly encompasses conservatively modified variants thereof.

In some embodiments, the linker is comprised a succession of at least 1, 2, 3, 4, 5, 6, 7; 8, 9, 10 Tumor-Associated Antigens (TAA) (e.g. NY-ESO-1, MelanA antigen, MUC-1).

In some embodiments, the linker is comprised a succession of at least 1, 2, 3, 4, 5, 6, 7; 8, 9, 10 Tolerance Antigens (e.g. MBP, PLP, MOG).

In some embodiments, the linker is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 1 1 , about 10, about 9, about 8, about 7, about 6, about 5, about 4, about 3, or about 2 amino acids long. In some embodiments, the linker is flexible. In another embodiment, the linker is rigid.

In some embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In some embodiments, the linker is a hinge region of an antibody (e.g., of IgG, IgA, IgD, and IgE, inclusive of subclasses (e.g., lgG1 , lgG2, lgG3, and lgG4, and lgA1 and lgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of lgG1 encompasses amino acids 216-231 and, because it is freely flexible, the Fab fragments can rotate about their axes of symmetry and move within a sphere centered at the first of two inter-heavy chain disulfide bridges. lgG2 has a shorter hinge than lgG1, with 12 amino acid residues and four disulfide bridges. The hinge region of lgG2 lacks a glycine residue, is relatively short, and contains a rigid poly-proline double helix, stabilized by extra inter-heavy chain disulfide bridges. These properties restrict the flexibility of the lgG2 molecule. lgG3 differs from the other subclasses by its unique extended hinge region (about four times as long as the lgG1 hinge), containing 62 amino acids (including 21 prolines and 11 cysteines), forming an inflexible poly-proline double helix. In lgG3, the Fab fragments are relatively far away from the Fc fragment, giving the molecule a greater flexibility. The elongated hinge in lgG3 is also responsible for its higher molecular weight compared to the other subclasses. The hinge region of lgG4 is shorter than that of lgG1 and its flexibility is intermediate between that of lgG1 and lgG2. The flexibility of the hinge regions reportedly decreases in the order lgG3>1gG1>lgG4>lgG2. In some embodiments, the linker may be derived from human lgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al./1992 Immunological Reviews 130:87. The upper hinge region includes amino acids from the carboxyl end of Cm to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH2 domain and includes residues in CH2. Id. The core hinge region of wild-type human lgG1 contains the sequence Cys-Pro-Pro-Cys which, when dimerized by disulfide bond formation, results in a cyclic octapeptide believed to act as a pivot, thus conferring flexibility. In some embodiments, the present linker comprises, one, or two, or three of the upper hinge region, the core region, and the lower hinge region of any antibody (e.g., of IgG, IgA, IgD, and IgE, inclusive of subclasses (e.g., lgG1 , lgG2, lgG3, and lgG4, and lgA1 and lgA2)). The hinge region may also contain one or more glycosylation sites, which include a number of structurally distinct types of sites for carbohydrate attachment. For example, lgAl contains five glycosylation sites within a 17-amino-acid segment of the hinge region, conferring resistance of the hinge region polypeptide to intestinal proteases, considered an advantageous property for a secretory immunoglobulin. In some embodiments, the linker of the present invention comprises one or more glycosylation sites.

In some embodiments, the linker comprises an Fc domain of an antibody (e.g., of IgG, IgA, IgD, and IgE, inclusive of subclasses (e.g., lgG1 , lgG2, lgG3, and lgG4, and lgA1 and lgA2)). In some embodiments, the linker comprises a hinge-CH2—CH3 Fc domain derived from a human lgG4 antibody. In some embodiments, the linker comprises a hinge-CH2—CH3 Fc domain derived from a human lgG1 antibody. In some embodiments, the Fc domain exhibits increased affinity for and enhanced binding to the neonatal Fc receptor (FcRn). In some embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In some embodiments, the linker does not comprise an Fc domain of an antibody.

As used herein, the term “personalized therapy” relates to information about a person's own genes or proteins to prevent, diagnose, or treat disease. In cancer, personalized medicine uses specific information about a person's tumor to help make a diagnosis, plan treatment, find out how well treatment is working, or make a prognosis.

In some embodiments, the linker (b) can be interchanged to adapt to the patient for a personalized therapy perspective. The antigens expressed by tumors or pathogenic microorganisms like bacteria and viruses can be inserted as antigenic peptides in the linker (b) of the chimeric protein of the invention. These chimeric proteins can thus be ‘personalized’ for each patient by changing the antigenic peptides of the linker (b) using sequences from the patient's specific antigens.

### Signaling Domain (a) or (c)

In some embodiments, the chimeric protein of the invention is composed of a signaling domain (a) and/or a signaling domain (c).

In some embodiments, the chimeric protein of the invention is composed of a monomeric signaling domain (a) or a dimeric (e.g. homodimeric or heterodimeric) signaling domain (a) or trimeric (e.g. homotrimeric or heterotrimeric) signaling domain (a). In some embodiments, the chimeric protein of the invention is composed of a monomeric signaling domain (c) or a dimeric signaling domain (c) or trimeric signaling domain (c).

As used herein, the term “signaling domain” refers to a domain that binds a surface molecule and initiates an intracellular signal transduction cascade. The ‘signaling domain’ is composed of monomeric, dimeric (e.g. homodimeric or heterodimeric) or trimeric (e.g. homotrimeric or heterotrimeric) proteins of the TNF family and possesses agonistic properties to stimulate the activation of different immune cell types.

In some embodiments, the signaling domain (c) or the signaling domain (a), in the case of the molecules from the Tumor Necrosis Factor Superfamily (TNFSF), comprises for instance but not limited to CD40L (CD154), 4-1BBL (CD137L) and OX40L (CD252), the active form being a homotrimer of the extracellular domain of the ligand. The TNFSF is composed of 27 members.

As used herein, the term “Tumor Necrosis Factor Superfamily” (TNFSF) refers to a protein superfamily of cytokine receptors characterized by the ability to bind tumor necrosis factors (TNFs) via an extracellular cysteine-rich domain. The TNFSF is composed of 27 members among them CD40L (CD154), 4-1BBL (CD137L) and OX40L (CD252), the active form being a homotrimer of the extracellular domain of the ligand.

As used herein, the term “Cluster of differentiation 40” (CD40) refers to a costimulatory protein found on antigen-presenting cells and is required for their activation. The binding of CD154 (CD40L) on TH cells to CD40 activates antigen presenting cells and induces a variety of downstream effects (UniProt number: P25942).

As used herein, the term “Cluster of differentiation 137 L” (CD137L), also known as 4-1BBL, necrosis factor receptor superfamily member 9 (TNFRSF9) and induced by lymphocyte activation (ILA). 4-1BBL is a member of the tumor necrosis factor (TNF) receptor family and is expressed by activated T cells of both the CD4+ and CD8+ lineages. 4-1 BB Lis also expressed on dendritic cells, B cells, NK cells, neutrophils and macrophages (UniProt number: Q07011). CD137 ligand is mainly expressed on professional antigen-presenting cells (APCs) such as dendritic cells, monocytes/macrophages, and B cells, and its expression is upregulated during activation of these cells. However, its expression has been documented on a variety of hematopoietic cells and non-hematopoietic cells. Generally, 4-1 BBL/CD137L is constitutively expressed on many types of cells but its expression levels are low except for a few types of cells. Interestingly, 4-1 BBL/CD137L is coexpressed with CD137 (also known as 4-1 BB and TNFRSF9) on various types of cells, but expression of CD137/4-1 BB potently downregulates that of 4-1 BBL/CD137L by cis-interactions between the two molecules resulting in endocytosis of 4-1 BBL/CD137L (see Byungsuk Kwon et al. Is CD137 Ligand (CD137L) “Signaling a Fine Tuner of Immune Responses?” Immune Netw. 2015 June; 15(3):121-124).

As used herein, the term “Tumor necrosis factor receptor superfamily, member 4”(TNFRSF4), also known as CD134 and OX40 receptor, is a member of the TNFR-superfamily of receptors which is not constitutively expressed on resting naïve T cells, unlike CD28 (UniProt number: P43489).

In some embodiments, the signaling domain (c) or the signaling domain (a) acts as a specific agonist for receptors expressed on the immune cells of interest (e.g. dendritic cells, T cells including Tregs, macrophages, NK cells, myeloid-derived suppressor cells) depending on the pathological context. The signaling domain (c) or the signaling domain (a) consists in an active form (monomeric or multimeric) of the extracellular domains of immune ligands (e.g. TNFSF family, PD-L1, CTLA-4).

As used herein the term “dendritic cells” (DCs) refer to antigen-presenting cells (also known as accessory cells) of the mammalian immune system. Their main function is to process antigen material and present it on the cell surface to the T cells of the immune system. They act as messengers between the innate and the adaptive immune systems.

As used herein the term “T cells” refers to a type of lymphocyte. T cells are one of the important white blood cells of the immune system and play a central role in the adaptive immune response. T cells can be easily distinguished from other lymphocytes by the presence of a T-cell receptor (TCR) on their cell surface. T cells are born from hematopoietic stem cells, found in the bone marrow. Then, developing T cells migrate to the thymus gland to mature. T cells derive their name from this organ where they develop (or mature). After migration to the thymus, the precursor cells mature into several distinct types of T cells. T cell differentiation also continues after they have left the thymus. Groups of specific, differentiated T cell subtypes have a variety of important functions in controlling and shaping the immune response. One of these functions is immune-mediated cell death, and it is carried out by two major subtypes: CD8+ “killer” and CD4+ “helper” T cells. (These are named for the presence of the cell surface proteins CD8 or CD4.) CD8+ T cells, also known as “killer T cells”, are cytotoxic—this means that they are able to directly kill virus-infected cells, as well as cancer cells. CD8+ T cells are also able to use small signaling proteins, known as cytokines, to recruit other types of cells when mounting an immune response. A different population of T cells, the CD4+ T cells, function as “helper cells”. Unlike CD8+ killer T cells, these CD4+ helper T cells function by indirectly killing cells identified as foreign: they determine if and how other parts of the immune system respond to a specific, perceived threat. Helper T cells also use cytokine signaling to influence regulatory B cells directly, and other cell populations indirectly.

As used herein the term “The regulatory T cells” (Tregs), formerly known as suppressor T cells refers to a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FOXP3, and CD25 and are thought to be derived from the same lineage as naïve CD4 cells.

As used herein the term “Natural killer cells” (NK cells) also known as large granular lymphocytes (LGL) refers to a type of cytotoxic lymphocyte critical to the innate immune system that belong to the rapidly expanding family of innate lymphoid cells (ILC) and represent 5-20% of all circulating lymphocytes in humans. The role of NK cells is analogous to that of cytotoxic T cells in the vertebrate adaptive immune response. NK cells provide rapid responses to virus-infected cells, acting at around 3 days after infection, and respond to tumor formation. Typically, immune cells detect the major histocompatibility complex (MHC) presented on infected cell surfaces, triggering cytokine release, causing the death of the infected cell by lysis or apoptosis. NK cells are unique, however, as they have the ability to recognize and kill stressed cells in the absence of antibodies and MHC, allowing for a much faster immune reaction. They were named “natural killers” because of the notion that they do not require activation to kill cells that are missing “self” markers of MHC class 1. This role is especially important because harmful cells that are missing MHC I markers cannot be detected and destroyed by other immune cells, such as T lymphocyte cells. NK cells can be identified by the presence of CD56 and the absence of CD3 (CD56+, CD3−). NK cells (belonging to the group of innate lymphoid cells) are one of the three kinds of cells differentiated from the common lymphoid progenitor, the other two being B and T lymphocytes. NK cells are known to differentiate and mature in the bone marrow, lymph nodes, spleen, tonsils, and thymus, where they then enter into the circulation.

As used herein the term “myeloid-derived suppressor cells” (MDSC) are a heterogeneous group of immune cells from the myeloid lineage (a family of cells that originate from bone marrow stem cells). MDSCs strongly expand in pathological situations such as chronic infections and cancer, as a result of an altered haematopoiesis. MDSCs are discriminated from other myeloid cell types in which they possess strong immunosuppressive activities rather than immunostimulatory properties. Similar to other myeloid cells, MDSCs interact with other immune cell types including T cells, dendritic cells, macrophages and natural killer cells to regulate their functions.

In some embodiments, the chimeric protein of the present invention comprises a trimer of extracellular domains of immune stimulatory signals which is are one or more of 4-1 BBL, OX-40 ligand (OX-40L), LIGHT (CD258), GITR ligand (GITRL), CD70, CD30 ligand, CD40 ligand (CD40L), CD137 ligand, TRAIL, and TL1A.

In a particular embodiment, the chimeric protein of the present invention comprises a combination of three identical immune stimulatory signals, wherein the immune stimulatory signals are chosen from, but are not limited to, 4-1 BBL, OX-40 ligand (OX-40L), LIGHT (CD258), GITR ligand (GITRL), CD70, CD30 ligand, CD40 ligand (CD40L), CD137 ligand, TRAIL, and TL1A.

In a particular embodiment, the chimeric protein of the present invention comprises a combination of three different immune stimulatory signals, wherein the immune stimulatory signals are chosen from, but are not limited to, 4-1 BBL, OX-40 ligand (OX-40L), LIGHT (CD258), GITR ligand (GITRL), CD70, CD30 ligand, CD40 ligand (CD40L), CD137 ligand, TRAIL, and TL1A.

In a particular embodiment, the chimeric protein of the present invention comprises a combination of two identical immune stimulatory signals and one different immune stimulatory signals, , wherein the immune stimulatory signals are chosen from, but are not limited to, 4-1 BBL, OX-40 ligand (OX-40L), LIGHT (CD258), GITR ligand (GITRL), CD70, CD30 ligand, CD40 ligand (CD40L), CD137 ligand, TRAIL, and TL1A.

In some embodiments, the chimeric protein of the present invention comprises a trimer of extracellular domains of immune stimulatory signals modified to improve their folding or ligand/receptor interactions.

As used herein, the term “LIGHT”, also known as tumor necrosis factor superfamily member 14 (TNFSF14), is a secreted protein of the TNF superfamily. It is recognized by herpesvirus entry mediator (HVEM), as well as decoy receptor 3 (UniProt number: 043557).

As used herein, the term “Tumor necrosis factor ligand superfamily member 18” (TNFSF18) also known as GITR ligand (GITRL) refers to a protein that in humans is encoded by the TNFSF18 gene (UniProt number: Q9UNG2).

As used herein, the term “Cluster of Differentiation 70” (CD70) refers to a protein that in humans is encoded by CD70 gene. [1] CD70 is a ligand for CD27. The CD70 protein is expressed on highly activated lymphocytes (like in T-and B-cell lymphomas). (UniProt number: P32970).

As used herein, the term “CD30 ligand” also known as TNFRSF8, is a cell membrane protein of the tumor necrosis factor receptor family and tumor marker. The protein encoded by this gene is a cytokine that belongs to the tumor necrosis factor (TNF) ligand family. This cytokine is a ligand for receptor TNFRSF18/AITR/GITR. It has been shown to modulate T lymphocyte survival in peripheral tissues. This cytokine is also found to be expressed in endothelial cells, and is thought to be important for interaction between T lymphocytes and endothelial cells. (UniProt number: P28908).

As used herein, the term “TNF-related apoptosis-inducing ligand (TRAIL) also been designated CD253 (cluster of differentiation 253) and TNFSF10 (tumor necrosis factor (ligand) superfamily, member 10) refers a protein functioning as a ligand that induces the process of cell death called apoptosis. TRAIL is a cytokine that is produced and secreted by most normal tissue cells. It causes apoptosis primarily in tumor cells, by binding to certain death receptors. (UniProt number: P50591).

As used herein, the term “Tumor necrosis factor ligand superfamily member 15” (TNFSF15) also known as TL1A refers to a cytokine that belongs to the tumor necrosis factor (TNF) ligand family. It is specifically expressed in endothelial cells (UniProt number: O95150).

As used herein the term “trimeric protein” refers to a macromolecular complex formed by three, usually non-covalently bound, macromolecules like proteins or nucleic acids. A homotrimer would be formed by three identical molecules. A heterotrimer would be formed by three different macromolecules. In particular, the term “trimeric” refers to a repetition (×3) of the sequence of the extracellular domain of each of these molecules and allowing optimal activation of the cells of interest through efficient binding to their trimerized cognate receptors.

In some embodiments, the signaling domain (c) or the signaling domain (a) is a trimeric protein. In some embodiments, the signaling domain (a) or (c) of the invention comprises a trimeric immune stimulatory agent as follows: trimeric 4-1 BBL; trimeric OX-40L; trimeric LIGHT; trimeric GITRL; trimeric CD70; trimeric CD30Ltrimeric CD40L; trimeric HVEM, trimeric GITR; trimeric CD27; trimeric CD28, trimeric CD137 and trimeric TL1A.

In a particular embodiment, the signaling domain of the invention comprises a trimeric 4-1 BBL.

In a particular embodiment, the signaling domain of the invention comprises three monomeric 4-1 BBL linked together by a linker, wherein the linker is selected from a repeats of 4 glycines and 1 serine (G4S sequences), a peptide tags for protein detection with a specific antibody by common laboratory techniques, one or several identified and therapeutically-relevant antigenic peptide sequences that can be presented on class I and class II molecules of the major histocompatibility complex and against which will be directed a specific immune response, for instance Tumor-Associated Antigens (TAA) (e.g. NY-ESO-1, MelanA antigen, MUC-1) or Tolerance Antigens (e.g. MBP, PLP, MOG). In a particular embodiment, the linker between the monomer of 4-1BBL is TAA.

In some embodiments, the signaling domain of the invention comprises a trimeric immune stimulatory agent with two monomers of 4-1 BBL and one monomeric OX-40L, wherein each monomers of 4-1 BBL are linked together by a linker, wherein the linker is selected from a repeats of 4 glycines and 1 serine (G4S sequences), a peptide tags for protein detection with a specific antibody by common laboratory techniques, one or several identified and therapeutically-relevant antigenic peptide sequences that can be presented on class I and class II molecules of the major histocompatibility complex and against which will be directed a specific immune response, for instance Tumor-Associated Antigens (TAA) (e.g. NY-ESO-1, MelanA antigen, MUC-1) or Tolerance Antigens (e.g. MBP, PLP, MOG). In a particular embodiment, the linker between the monomer of 4-1L is TAA.

In a particular embodiment, the signaling domain of the invention comprises a trimeric OX-40L. In a particular embodiment, the signaling domain of the invention comprises three monomeric OX-40L linked together by a linker, wherein the linker is selected from a repeats of 4 glycines and 1 serine (G4S sequences), a peptide tags for protein detection with a specific antibody by common laboratory techniques, one or several identified and therapeutically-relevant antigenic peptide sequences that can be presented on class I and class II molecules of the major histocompatibility complex and against which will be directed a specific immune response, for instance Tumor-Associated Antigens (TAA) (e.g. NY-ESO-1, MelanA antigen, MUC-1) or Tolerance Antigens (e.g. MBP, PLP, MOG). In a particular embodiment, the linker between the monomer of OX-40L is TAA.

In a particular embodiment, the signaling domain of the invention comprises a trimeric CD40L. In a particular embodiment, the signaling domain of the invention comprises three monomeric CD40L linked together by a linker, wherein the linker is selected from a repeats of 4 glycines and 1 serine (G4S sequences), a peptide tags for protein detection with a specific antibody by common laboratory techniques, one or several identified and therapeutically-relevant antigenic peptide sequences that can be presented on class I and class II molecules of the major histocompatibility complex and against which will be directed a specific immune response, for instance Tumor-Associated Antigens (TAA) (e.g. NY-ESO-1, MelanA antigen, MUC-1) or Tolerance Antigens (e.g. MBP, PLP, MOG). In a particular embodiment, the linker between the monomer of CD40L is TAA.

As used herein, the term “trimeric” relates to a chemical compound or molecule consisting of three identical simpler molecules or three different simpler molecules, or two identical simpler molecules and one different simpler molecule. In some embodiment, the term “trimeric” includes homotrimeric or heterotrimeric.

In some embodiments, the signaling domain (c) or the signaling (a) is monomeric. As used herein, the term “monomeric” relates to a chemical compound or molecule consisting of one simpler molecule.

In some embodiments, the signaling domain (c) or the signaling (a) is dimeric.

As used herein, the term “dimeric” relates to a chemical compound or molecule consisting of two identical simpler molecule or two different simpler molecule. In some embodiment, the term “dimeric” includes homodimeric or heterodimeric.

### The Vectorization of the Proteins

In some embodiments, the present invention relates to a vector for delivery of a heterologous nucleic acid, wherein the nucleic acid encodes the chimeric protein of the present invention.

In some embodiments, the vectorization of the nucleic acid encoding the chimeric protein or of the chimeric protein of the present invention is used any expression system, and is not limited to replicating and non-replicating viruses (e.g. oncolytic viruses, AAV, lentiviruses), mRNA, plasmids and other gene therapy vectors.

In some embodiments, the present invention relates to a vesicular stomatitis virus (VSV) comprising a nucleic acid encoding the chimeric protein having a general structure of N terminus-(a)-(b)-(c)-C terminus wherein (a) is a domain, (b) is a functional linker and (c) is a domain.

In some embodiments, the present invention relates to a vesicular stomatitis virus (VSV) comprising a nucleic acid encoding the chimeric protein having a general structure of N terminus-(c)-(b)-(a)-C terminus wherein (a) is a domain, (b) is a functional linker and (c) is a domain.

In some embodiments, the present invention relates to vesicular stomatitis virus (VSV) comprising a nucleic acid encoding the chimeric protein having a general structure of N terminus-(a)-(b)-(c)-C terminus wherein (a) is a monomeric domain, (b) is a functional linker and (c) is a trimeric domain.

In some embodiments, the present invention relates to vesicular stomatitis virus (VSV) comprising a nucleic acid encoding the chimeric protein having a general structure of N terminus-(c)-(b)-(a)-C terminus wherein (c) is a monomeric domain, (b) is a functional linker and (a) is a trimeric domain.

In some embodiments, the present invention relates to vesicular stomatitis virus (VSV) comprising a nucleic acid encoding the chimeric protein having a general structure of N terminus-(a)-(b)-(c)-C terminus wherein (a) is a monomeric domain, (b) is a functional linker and (c) is a dimeric domain.

In some embodiments, the present invention relates to vesicular stomatitis virus (VSV) comprising a nucleic acid encoding the chimeric protein having a general structure of N terminus-(c)-(b)-(a)-C terminus wherein (c) is a monomeric domain, (b) is a functional linker and (a) is a dimeric domain.

In some embodiments, the present invention relates to a vesicular stomatitis virus (VSV) comprising a chimeric protein having a general structure of N terminus-(a)-(b)-(c)-C terminus wherein (a) is a domain, (b) is a functional linker and (c) is a domain.

In some embodiments, the present invention relates to a vesicular stomatitis virus (VSV) comprising a chimeric protein having a general structure of N terminus-(c)-(b)-(a)-C terminus wherein (a) is a domain, (b) is a functional linker and (c) is a domain.

In some embodiments, the present invention relates to vesicular stomatitis virus (VSV) comprising a chimeric protein having a general structure of N terminus-(a)-(b)-(c)-C terminus wherein (a) is a monomeric domain, (b) is a functional linker and (c) is a trimeric domain.

In some embodiments, the present invention relates to vesicular stomatitis virus (VSV) comprising a chimeric protein having a general structure of N terminus-(c)-(b)-(a)-C terminus wherein (c) is a monomeric domain, (b) is a functional linker and (a) is a trimeric domain.

In some embodiments, the present invention relates to vesicular stomatitis virus (VSV) comprising a chimeric protein having a general structure of N terminus-(a)-(b)-(c)-C terminus wherein (a) is a monomeric domain, (b) is a functional linker and (c) is a dimeric domain.

In some embodiments, the present invention relates to vesicular stomatitis virus (VSV) comprising a chimeric protein having a general structure of N terminus-(c)-(b)-(a)-C terminus wherein (c) is a monomeric domain, (b) is a functional linker and (a) is a dimeric domain.

In some embodiments, the present invention relates to a vaccinia virus (VV) comprising a nucleic acid encoding the chimeric protein having a general structure of N terminus-(a)-(b)-(c)-C terminus wherein (a) is a domain, (b) is a functional linker and (c) is a domain.

In some embodiments, the present invention relates to a vaccinia virus (VV) comprising a nucleic acid encoding the chimeric protein having a general structure of N terminus-(c)-(b)-(a)-C terminus wherein (a) is a domain, (b) is a functional linker and (c) is a domain.

In some embodiments, the present invention relates to vaccinia virus (VV) comprising a nucleic acid encoding the chimeric protein having a general structure of N terminus-(a)-(b)-(c)-C terminus wherein (a) is a monomeric domain, (b) is a functional linker and (c) is a trimeric domain.

In some embodiments, the present invention relates to vaccinia virus (VV) comprising a nucleic acid encoding the chimeric protein having a general structure of N terminus-(c)-(b)-(a)-C terminus wherein (c) is a monomeric domain, (b) is a functional linker and (a) is a trimeric domain.

In some embodiments, the present invention relates to vaccinia virus (VV) comprising a nucleic acid encoding the chimeric protein having a general structure of N terminus-(a)-(b)-(c)-C terminus wherein (a) is a monomeric domain, (b) is a functional linker and (c) is a dimeric domain.

In some embodiments, the present invention relates to vaccinia virus (VV) comprising a nucleic acid encoding the chimeric protein having a general structure of N terminus-(c)-(b)-(a)-C terminus wherein (c) is a monomeric domain, (b) is a functional linker and (a) is a dimeric domain.

In some embodiments, the present invention relates to a vaccinia virus (VV) comprising a nucleic acid encoding the chimeric protein having a general structure of N terminus-(a)-(b)-(c)-C terminus wherein (a) is a domain, (b) is a functional linker and (c) is a domain.

In some embodiments, the present invention relates to a vaccinia virus (VV) comprising a nucleic acid encoding the chimeric protein having a general structure of N terminus-(c)-(b)-(a)-C terminus wherein (a) is a domain, (b) is a functional linker and (c) is a domain.

In some embodiments, the present invention relates to vaccinia virus (VV) comprising a nucleic acid encoding the chimeric protein having a general structure of N terminus-(a)-(b)-(c)-C terminus wherein (a) is a monomeric domain, (b) is a functional linker and (c) is a trimeric domain.

In some embodiments, the present invention relates to vaccinia virus (VV) comprising a nucleic acid encoding the chimeric protein having a general structure of N terminus-(c)-(b)-(a)-C terminus wherein (c) is a monomeric domain, (b) is a functional linker and (a) is a trimeric domain.

In some embodiments, the present invention relates to vaccinia virus (VV) comprising a nucleic acid encoding the chimeric protein having a general structure of N terminus-(a)-(b)-(c)-C terminus wherein (a) is a monomeric domain, (b) is a functional linker and (c) is a dimeric domain.

In some embodiments, the present invention relates to vaccinia virus (VV) comprising a nucleic acid encoding the chimeric protein having a general structure of N terminus-(c)-(b)-(a)-C terminus wherein (c) is a monomeric domain, (b) is a functional linker and (a) is a dimeric domain.

In some embodiments, the present invention provides an expression vector, comprising a nucleic acid encoding the chimeric protein described herein. In some embodiments, the expression vector comprises DNA or RNA. In some embodiments, the expression vector is a mammalian expression vector.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and APL. Non-limiting examples of prokaryotic expression vectors may include the Agt vector series such as Agt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the chimeric proteins in recombinant host cells.

In some embodiments, the expression vectors of the invention comprise a nucleic acid encoding the chimeric proteins (and/or additional agents), or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector. Expression control regions are regulatory polynucleotides (sometimes referred to herein as elements), such as promoters and enhancers, that influence expression of an operably linked nucleic acid. An expression control region of an expression vector of the invention is capable of expressing operably linked encoding nucleic acid in a human cell. In some embodiment, the cell is a tumor cell. In another embodiment, the cell is a non-tumor cell. In some embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression. In some embodiment, the present invention contemplates the use of inducible promoters capable of effecting high level of expression transiently in response to a cue. For example, when in the proximity of a tumor cell, a cell transformed with an expression vector for the chimeric protein (and/or additional agents) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941 , each of which is incorporated herein by reference in its entirety. Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

In some embodiments the chimeric protein of the invention is or can be secreted.

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter. Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs. There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al, J. Biol. Chem. 262, 4429-4432 (1987); and Wagner et al., Proc. Natl. Acad. Sci. USA 87, 3410-3414 (1990). Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacterid., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton,

J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al, J. Mol. Biol. 335:667-678, 2004), sleeping beauty, transposases of the mariner family (Plasterk et al., supra), and components for integrating viruses such as AAV, retroviruses, lentivirus having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric fusion proteins including CRISPR/CAS9, zinc finger, TALEN, and meganuclease gene-editing technologies.

In one aspect, the invention provides expression vectors for the expression of the chimeric proteins (and/or additional agents) that are viral vectors. Many viral vectors useful for gene therapy are known (see, e.g., Lundstrom, Trends Biotechnol., 21: 1 17, 122, 2003. Illustrative viral vectors include those selected from vesicular stomatitis virus (VSV), lentivirus, (LV), retroviruses (RV), adenoviruses (AV), adeno-associated viruses (AAV) or a virus from the same families, measles virus, herpes virus, vaccinia virus (VV), myxoma virus, reovirus, parvovirus, mumps virus and a viruses though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV), HSV, enterovirus, paramyxovirus, poxvirus, rhabdovirus. For in vitro uses, viral vectors that integrate into the host genome are suitable, such as retroviruses, AAV, lentivirus. In some embodiments, the invention provides methods of transducing a human cell in vivo, comprising contacting a solid tumor in vivo with a viral vector of the invention.

In a particular embodiment, the viral vector of the invention is a vesicular stomatitis virus (VSV).

In a particular embodiment, the viral vector of the invention is a recombinant vaccinia virus (VV).

In some embodiments, the present invention provides a host cell, comprising the expression vector comprising the chimeric protein described herein.

Expression vectors can be introduced into host cells for producing the present chimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 {e.g., COS-7, ATCC CRL 1651); human embryonic kidney lines {e.g., 293, 293-EBNA, or 293 cells subcloned for growth in suspension culture, Graham et al., J Gen Virol 1977, 36:59); baby hamster kidney cells {e.g., BHK, ATCC CCL 10); Chinese hamster ovary-cells-DHFR {e.g., CHO, Urlaub and Chasin, Proc Natl Acad Sci USA 1980, 77:4216); DG44 CHO cells, CHO-K1 cells, mouse Sertoli cells (Mather, Biol Reprod 1980, 23:243-251); mouse fibroblast cells {e.g., NIH-3T3), monkey kidney cells (e.g., CVI ATCC CCL 70); African green monkey kidney cells, (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the chimeric proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction.

Host cells can be obtained from normal or affected subjects, including healthy humans, cancer patients, and patients with an infectious disease, private laboratory deposits, public culture collections such as the American Type Culture Collection, or from commercial suppliers.

Cells that can be used for production of the present chimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, fetal liver, etc. The choice of cell type depends on the type of tumor or infectious disease being treated or prevented, and can be determined by one of skill in the art.

### Treatment

In some embodiments, the present invention relates to a method of treatment or prevention of cancers and/or tumors. The treatment of cancer may involve modulating the immune system with the present chimeric proteins to favor immune stimulation over immune inhibition.

In some embodiments, the present invention relates to a chimeric protein for use in a method of treatment or prevention in a patient suffering from cancer.

In some embodiments, the present invention relates to a method of treatment of a subject suffering from cancer comprising administered a therapeutically effective amount of a chimeric protein comprising the structure: N terminus-(a)-(b)-(c)-C terminus wherein (a) is a domain, (b) is a functional linker and (c) is a domain.

In some embodiments, the present invention relates to a method of treatment of a subject suffering from cancer comprising administered a therapeutically effective amount of a chimeric protein comprising the structure: N terminus-(c)-(b)-(a)-C terminus wherein (a) is a domain, (b) is a functional linker and (c) is a domain.

In some embodiments, the present invention relates to a method of treatment of a subject suffering from cancer comprising administered a therapeutically effective amount of a chimeric protein comprising the structure: N terminus-(a)-(b)-(c)-C terminus wherein (a) is a signaling and/or targeting domain, (b) is a functional linker and (c) is a signaling and/or targeting domain.

In some embodiments, the present invention relates to a method of treatment of a subject suffering from cancer comprising administered a therapeutically effective amount of a chimeric protein comprising the structure: N terminus-(c)-(b)-(a)-C terminus wherein (a) is a signaling and/or targeting domain, (b) is a functional linker and (c) is a signaling and/or targeting domain.

In some embodiments, the present invention relates to a method of treatment of a subject suffering from cancer comprising administered a therapeutically effective amount of a chimeric protein comprising the structure: N terminus-(a)-(b)-(c)-C terminus wherein (a) is a monomeric domain, (b) is a functional linker and (c) is a trimeric domain.

In some embodiments, the present invention relates to a method of treatment of a subject suffering from cancer comprising administered a therapeutically effective amount of a chimeric protein comprising the structure: N terminus-(c)-(b)-(a)-C terminus wherein (c) is a monomeric domain, (b) is a functional linker and (a) is a trimeric domain.

In some embodiments, the present invention relates to a method of treatment of a subject suffering from cancer comprising administered a therapeutically effective amount of a chimeric protein comprising the structure: N terminus-(a)-(b)-(c)-C terminus wherein (a) is a monomeric domain, (b) is a functional linker and (c) is a dimeric domain.

In some embodiments, the present invention relates to a method of treatment of a subject suffering from cancer comprising administered a therapeutically effective amount of a chimeric protein comprising the structure: N terminus-(c)-(b)-(a)-C terminus wherein (c) is a monomeric domain, (b) is a functional linker and (a) is a dimeric domain.

As used herein, the term “subject” denotes a mammal, such as a rodent, a feline, a canine, and a primate. Particularly, the subject according to the invention is a human. More particularly, the subject according to the invention has or is susceptible to have cancer.

As used herein, the term “subject” encompasses “patient”.

As used herein, the term “biological sample” refers to any sample obtained from a subject, such as a serum sample, a plasma sample, a urine sample, a blood sample, a lymph sample, or a tissue biopsy. In a particular embodiment, the biological sample is a tissue biopsy.

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

In some embodiments, when the subject suffers from a cancer, the tissue sample is a tumor tissue sample. As used herein, the term “tumor tissue sample” means any tissue tumor sample derived from the subject. Said tissue sample is obtained for the purpose of the in vitro evaluation. In some embodiments, the tumor sample may result from the tumor resected from the subject. In some embodiments, the tumor sample may result from a biopsy performed in the primary tumor of the subject or performed in metastatic sample distant from the primary tumor of the subject. In some embodiments, the tumor tissue sample encompasses a global primary tumor (as a whole), a tissue sample from the center of the tumor, a tumor tissue sample collected prior surgery (for follow-up of subjects after treatment for example), and a distant metastasis. The tumor tissue sample can, of course, be subjected to a variety of well-known post-collection preparative and storage techniques (e.g., fixation, storage, freezing, etc.). The sample can be fresh, frozen, fixed (e.g., formalin fixed), or embedded (e.g., paraffin embedded.

As used herein, the terms “cancer” has its general meaning in the art and refers to a group of diseases involving abnormal cell growth with the potential to invade or spread to other parts of the body. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; non encapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brennertumor, malignant; phyllodestumor, malignant; synovial sarcoma; mesothelioma, malignant; dysgerminoma; embryonal carcinoma; teratoma, malignant; strumaovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblasticodontosarcoma; ameloblastoma, malignant; ameloblasticfibrosarcoma; pinealoma, malignant; chordoma; glioma, malignant; ependymoma; ependymoma; protoplasmic astrocytoma; fibrillary astrocytoma; astroblastoma; medulloblastoma, glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocyticleukemia; mast cell leukemia; megakaryoblasticleukemia; myeloid sarcoma; and hairy cell leukemia.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein the terms “administering” or “administration” refer to the act of injecting or otherwise physically delivering a substance as it exists outside the body (e.g., an inhibitor of caspase 8 alone or in a combination with a classical treatment) into the subject, such as by, intravenous, intramuscular, enteral, subcutaneous, parenteral, systemic, local, spinal, nasal, topical or epidermal administration (e.g., by injection or infusion). When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the chimeric protein or chimeric protein portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an chimeric protein of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non limiting examples, treatment according to the present invention may be provided as a daily dosage of the agent of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically, the protein or the gene coding for the chimeric protein of the invention as described above is administered to the subject in the form of a pharmaceutical composition which comprises a pharmaceutically acceptable carrier. Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. For use in administration to a subject, the composition will be formulated for administration to the subject. The compositions of the present invention may be administered orally, parenterally, by inhalation spray, topically, rectally, nasally, buccally, vaginally, intrapleurally, intraperitoneally or via an implanted reservoir. The used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include, e.g., lactose. When aqueous suspensions are required for oral use, the active ingredient is combined with emulsifying and suspending agents. If desired, certain sweetening, flavoring or coloring agents may also be added. Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols. The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Topical application for the lower intestinal tract can be effected in a rectal suppository formulation (see above) or in a suitable enema formulation. Patches may also be used. The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. For example, an chimeric protein present in a pharmaceutical composition of this invention can be supplied at a concentration of 10 mg/mL in either 100 mg (10 mL) or 500 mg (50 mL) single-use vials. The product is formulated for IV administration in 9.0 mg/mL sodium chloride, 7.35 mg/mL sodium citrate dihydrate, 0.7 mg/mL polysorbate 80, and Sterile Water for Injection. The pH is adjusted to 6.5. An exemplary suitable dosage range for an chimeric protein in a pharmaceutical composition of this invention may between about 1 mg/m2 and 500 mg/m2. However, it will be appreciated that these schedules are exemplary and that an optimal schedule and regimen can be adapted taking into account the affinity and tolerability of the particular antibody in the pharmaceutical composition that must be determined in clinical trials. A pharmaceutical composition of the invention for injection (e.g., intramuscular, i.v.) could be prepared to contain sterile buffered water (e.g. 1 ml for intramuscular), and between about 1 ng to about 100 mg, e.g. about 50 ng to about 30 mg or more preferably, about 5 mg to about 25 mg, of the inhibitor of the invention.

As used herein, the term “combination” is intended to refer to all forms of administration that provide a first drug together with a further (second, third . . . ) drug. The drugs may be administered simultaneous, separate or sequential and in any order. According to the invention, the drug is administered to the subject using any suitable method that enables the drug to reach the lungs. In some embodiments, the drug administered to the subject systemically (i.e. via systemic administration). Thus, in some embodiments, the drug is administered to the subject such that it enters the circulatory system and is distributed throughout the body. In some embodiments, the drug is administered to the subject by local administration, for example by local administration to the lungs.

As used herein, the term “combination” is intended to refer to all forms of administration that provide a first drug together with a further (second, third . . . ) drug. The present chimeric protein may be administered simultaneously, separately or sequentially and in any order. According to the invention, the present chimeric protein is administered to the subject using any suitable method that enables the drug to reach the kidney. In some embodiments, the present chimeric protein is administered to the subject systemically (i.e. via systemic administration). Thus, in some embodiments, the drug is administered to the subject such that it enters the circulatory system and is distributed throughout the body.

As used herein, the terms “combined treatment”, “combined therapy” or “therapy combination” refer to a treatment that uses more than one medication. The combined therapy may be dual therapy or bi-therapy.

In some embodiments, the present invention chemical compounds that are effective in inhibiting tumor growth. Examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin morpholino-doxorubicin, (including cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene;

edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1 ; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

As used herein the term “immunotherapeutic” as used herein refers to any type of compound which can be used in immunotherapy. Immunotherapy as used herein is the treatment of disease by inducing, enhancing, or suppressing an immune response. An “anti-cancer immunotherapy”, as used herein, stimulates the immune system to reject and destroy tumors. As used herein the term “anti-cancer immunotherapeutic” as used herein thus includes such compounds as e.g. Tumor-Specific Antigens (TSA), Tumor-Associated Antigens (TAA), immune adjuvants, immune modulators, antibodies, modified immune cells, cytokines, immune checkpoint blockade molecules, viruses.

As used herein the term “immune modulator”, as used herein, is a component that modulates the immune responses to an antigen towards the desired immune responses.

In some embodiments, the present invention includes additional agent which can be one or more immune-modulating agents. As used herein the term “immunotherapeutic agent” refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, immune checkpoint inhibitor, cytokines, cancer vaccines, monoclonal antibodies and non-cytokine adjuvants. Alternatively, the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), and IFN-beta (IFN-β). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include sargramostim. Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. In addition to having specific or non-specific targets, immunotherapeutic agents can be active, i.e. stimulate the body's own immune response, or they can be passive, i.e. comprise immune system components that were generated external to the body. Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins. Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies, anti-PDL1 antibodies, anti-PLD2 antibodies (selected by way of non-limiting example, one or more of nivolumab (ONO-4538/BMS-936558, MDX1 106, OPDIVO, BRISTOL MYERS SQUIBB), pembrolizumab (KEYTRUDA, Merck), MK-3475 (MERCK), BMS 936559 (BRISTOL MYERS SQUIBB), atezolizumab (TECENTRIQ, GENENTECH), MPDL3280A (ROCHE)), anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference], an agent that increases and/or stimulates CD137 (4-1 BB) and/or the binding of CD137 (4-1 BB) with one or more of 4-1 BB ligand (by way of non-limiting example, urelumab (BMS-663513 and anti-4-1 BB antibody), and an agent that blocks, reduces and/or inhibits the activity of CTLA-4 and/or the binding of CTLA-4 with one or more of AP2M 1, CD80, CD86, SHP-2, and PPP2R5A and/or the binding of OX40 with OX40L (by way of non-limiting example GBR 830 (GLENMARK), MEDI6469 (MEDIMMUNE).

The immunotherapeutic treatment may consist of allografting, in particular, allograft with hematopoietic stem cell HSC. The immunotherapeutic treatment may also consist in an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, NK cells, are isolated amplified in vitro and readministered to the subject. The activated lymphocytes or NK cells are most particularly be the subject's own cells that were earlier isolated from a blood or tumor sample and activated (or “expanded”) in vitro.

In some embodiments, the chimeric proteins are used to treat, control or prevent one or more inflammatory diseases or conditions. Non-limiting examples of inflammatory diseases include acne vulgaris, acute inflammation, allergic rhinitis, asthma, atherosclerosis, atopic dermatitis, autoimmune disease, autoinflammatory diseases, autosomal recessive spastic ataxia, bronchiectasis, celiac disease, chronic cholecystitis, chronic inflammation, chronic prostatitis, colitis, diverticulitis, familial eosinophilia (fe), glomerulonephritis, glycerol kinase deficiency, hidradenitis suppurativa, hypersensitivities, inflammation, inflammatory bowel diseases, inflammatory pelvic disease, interstitial cystitis, laryngeal inflammatory disease, Leigh syndrome, lichen planus, mast cell activation syndrome, mastocytosis, ocular inflammatory disease, otitis, pain, pelvic inflammatory disease, reperfusion injury, respiratory disease, restenosis, rheumatic fever, rheumatoid arthritis, rhinitis, sarcoidosis, septic shock, silicosis or other pneumoconioses, transplant rejection, tuberculosis, and vasculitis.

In some embodiments, the inflammatory disease is an autoimmune disease or condition, such as multiple sclerosis (e.g. autoimmune encephalomyelitis (EAE)), diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, or other autoimmune diseases.

In some embodiments, when the chimeric proteins of the invention are used to treat, control or prevent transplantation rejection, the chimeric proteins has a general structure of: N terminus-(a)-(b)-(c)-C terminus, wherein: (a) is a signaling and/or targeting domain, (b) is a linker (b) is a functional linker and (c) is a monomeric signaling and/or targeting domain or has a general structure of: N terminus-(c)-(b)-(a)-C terminus, wherein: (c) is a signaling and/or targeting domain, (b) is a linker (b) is a functional linker and (a) is a monomeric signaling and/or targeting domain.

In some embodiments, the present chimeric agents are used to eliminate intracellular pathogens. In some aspects, the present chimeric agents are used to treat one or more infections.

In some embodiments, the present chimeric proteins are used in methods of treating viral infections (including, for example, HIV and HCV), parasitic infections (including, for example, malaria), and bacterial infections. In some embodiments, the infections induce immunosuppression. For example, HIV infections often result in immunosuppression in the infected subjects. Accordingly, as described elsewhere herein, the treatment of such infections may involve, in some embodiments, modulating the immune system with the present chimeric proteins to favor immune stimulation over immune inhibition. Alternatively, the present invention provides methods for treating infections that induce immunoactivation. For example, intestinal helminth infections have been associated with chronic immune activation. In these embodiments, the treatment of such infections may involve modulating the immune system with the present chimeric proteins to favor immune inhibition over immune stimulation.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term“administration sequentially” refers to an administration of 2 active ingredients at different times, the administration route being identical or different.

In some embodiment, the chimeric protein of the invention may target a cell (e.g., cancer cell or immune cell) that expresses PD-L1 and/or PD-L2. In an illustrative embodiment, the chimeric protein may target a cell (e.g., cancer cell or immune cell) that expresses OX-40.

In some embodiment, the chimeric protein may target a cell (e.g., cancer cell or immune cell) that expresses GITR. In an illustrative embodiment, the chimeric protein may target a cell (e.g., cancer cell or immune cell) that expresses 4-1 BB. In some embodiment, the chimeric protein may target a cell (e.g., cancer cell or immune cell) that expresses CD40. In an illustrative embodiment, the chimeric protein may target a cell (e.g., cancer cell or immune cell) that expresses VISTA. In an illustrative embodiment, the chimeric protein may target a cell (e.g., cancer cell or immune cell) that expresses CSF1. In some embodiment, the chimeric protein may target a cell (e.g., cancer cell or immune cell) that expresses IL-34.

In some embodiment, the chimeric protein may target a cell (e.g., cancer cell or immune cell) that expresses CD47. In some embodiment, the chimeric protein may target a cell (e.g., cancer cell, stromal cell or immune cell) that expresses galectin-9 and/or phosphatidyserine. In some embodiments, the present methods provide treatment with the chimeric protein in a patient who is refractory to an additional agent, such “additional agents” being described elsewhere herein, inclusive, without limitation, of the various chemotherapeutic agents described herein.

In some embodiments, when the chimeric proteins of the invention are used to treat, control or prevent cancer or use in immunotherapy, the chimeric proteins has a general structure of: N terminus-(a)-(b)-(c)-C terminus, wherein: (a) is a signaling and/or targeting domain, (b) is a linker (b) is a functional linker and (c) is a dimeric or trimeric signaling and/or targeting domain or has a general structure of: N terminus-(c)-(b)-(a)-C terminus, wherein: (c) is a signaling and/or targeting domain, (b) is a linker (b) is a functional linker and (a) is a dimeric or trimeric signaling and/or targeting domain.

The invention will be further illustrated by the following figures and examples. However, these examples and figures should not be interpreted in any way as limiting the scope of the present invention.

### FIGURES

FIG. 1. General representation of the TAME-IT molecules. TAME-IT molecules are chimeric proteins composed of three different domains with distinct immunostimulating activities. The ‘targeting domain’ recognizes a marker on the surface of either tumor cells or immune cells; this anchors and displays the TAME-IT molecule on the cell surface and may signal or block signaling inside the targeted cells. The ‘functional linker’ contains peptide sequences of tumor or viral antigens that will be processed and presented on major histocompatibility complex molecules by different cell types, including by antigen-presenting cells upon phagocytosis of TAME-IT-targeted cells. The ‘signaling domain’ is composed of monomeric, dimeric or trimeric proteins of the TNF family and possesses agonistic properties to stimulate the activation of different immune cell types.

FIG. 2. TAME-IT proteins display agonistic ligands on the surface of targeted cells. PD-L1⁺ cells were incubated with the KN035-TAA-th4-1BBL molecule, KN035 being an anti-PD-L1 nanobody and th4-1BBL a trimeric human 4-1BBL domain, washed and then stained with a fluorochrome-conjugated anti-4-1BBL antibody. The TAME-IT molecule KN035-th4-1BBL is able to recognize surface PD-L1 and renders PD-L1⁺ cells positive for 4-1BBL (A). This process is more efficient than previous molecules composed of a human PD-1 moiety linked to a monomeric human 4-1BBL (mh4-1BBL) (D). Mesothelin⁺ cells were incubated with the A1-thCD40L molecule, A1 being an anti-mesothelin nanobody and thCD40L a trimeric human CD40L domain, washed and then stained with a fluorochrome-conjugated anti-CD40L antibody (B). The TAME-IT molecule A1-thCD40L is able to recognize surface mesothelin and renders mesothelin⁺ cells positive for CD40L (dark grey) while mesothelin⁺ cells stayed CD40L⁻ (light grey). When using a molecule composed only of the soluble trimeric CD40L (sthCD40L), mesothelin⁺ cells stayed CD40L⁻, which demonstrates that the anti-mesothelin A1 nanobody is necessary for surface fixation (C). CTLA-4⁺/CD40L⁻ cells were incubated (dark grey) or not (light grey) with an [Anti-CTLA-4]-[thCD40L] TAME-IT molecule, washed and then stained with a fluorochrome-conjugated anti-CD40L antibody. (E) The TAME-IT molecule renders CTLA-4⁺/CD40L⁻ cells positive for CD40L due to the fixation on their surface.

FIG. 3. TAME-IT molecules activate human dendritic cells. (A-C) Human monocyte-derived dendritic cells were incubated with the supernatants from either KN035-th4-1BBL-producing cells (dark grey) or control cells (light grey) for 48 h. Dendritic cells were then analyzed for the expression of CD80, CD83 and CD86 maturation markers by flow cytometry. This shows that the TAME-IT proteins induce the maturation of antigen-presenting cells. Human monocyte-derived dendritic cells (DC) were analyzed for the expression of the CD83 maturation marker after having been incubated for 20 h with LPS (positive control) and/or mesothelin+ tumor cells (Meso13) previously incubated or not with the TAME-IT molecule A1-thCD40L, A1 being an anti-mesothelin nanobody. The fixation of the TAME-IT molecule on tumor cells reverted the inhibition of DC maturation induced by the latter. When either a truncated version of the TAME-IT molecule lacking the anti-mesothelin nanobody (sthCD40L) was used or a physical separation between DC and tumor cells (transwell) was performed, this immunoactivating property was lost.

FIG. 4. Cells infected by engineered oncolytic viruses produce TAME-IT molecules. HEK cells were infected by with a VSV coding for the A1-thCD40L molecule. The culture supernatant (SN) was collected and concentrated by centrifugation (CSN). The A1-thCD40L was then detected by western-blot. As a negative control, a VSV coding for a non-relevant protein (mRuby2) was used.

FIG. 5. Antigens encoded in the ‘functional linker’ are efficiently presented to CD4 T cells. Human tumor cells were infected with a VSV coding for different TAME-IT molecules containing or not a ‘functional linker’ that contains antigenic peptides. These infected cells were then co-cultured or not with human monocyte-derived dendritic cells before being co-cultured for 6 hours with a human, HLA-DP4-restricted, CD4 T cell clone specific for the tumor antigen NY-ESO-1 which peptidic sequence is found in the ‘functional linker’. T cell activation was evaluated by intracellular staining of gamma-interferon and was only observed against HLA-DP4⁺ dendritic cells (expressing MHC class II molecules) that previously internalized tumor cells infected with the VSV coding for TAME-IT molecules containing a ‘functional linker’. This shows that the antigenic peptides from the TAME-IT molecules are efficiently processed by human antigen-presenting cells and presented to tumor-associated antigen-specific human CD4 T cells.

FIG. 6. Antigens encoded in the ‘functional linker’ are efficiently presented to CD8 T cells. Human tumor cells were infected with VSV encoding for different TAME-IT molecules containing or not a ‘functional linker’ that contains antigenic peptides. These infected cells were then co-cultured or not with human monocyte-derived dendritic cells before being co-cultured for 6 hours with a human, HLA-A2-restricted, CD8 T cell clone specific for the tumor antigen NY-ESO-1 which peptidic sequence is found in the ‘functional linker’. T cell activation was evaluated by intracellular staining of gamma-interferon and was only observed against HLA-A2⁺ tumor cells and dendritic cells in the conditions where a VSV coding for TAME-IT molecules containing a ‘functional linker’ was used. This shows that the antigenic peptides from the TAME-IT molecules are efficiently processed by infected cells or immune cells and presented to tumor-associated antigen-specific human CD8 T cells.

FIG. 7. TAME-IT proteins activate different types of human lymphocytes. (A) PD-L1⁺ human melanoma cells were co-cultured for 5 days with activated or non-activated human polyclonal CD8 T cells at target:effector ratios of 1:2, 1:1 or 2:1, in presence or not of the anti-PD-LI antibody nivolumab or the TAME-IT protein KN035-TAA-th4-1BBL. The confluence of the target melanoma cells was analyzed by microscopy during the experiment to evaluate killing by CD8 T cells. The TAME-IT protein enhances the activation of CD8 T cells and the killing of melanoma cells. (B) MDA-MB-231 human breast tumor cells were infected for 18 h with a VSV encoding or not for the KN035-TAA-th4-1BBL TAME-IT molecule. These infected cells were then co-cultured for 6h with human natural killer (NK) cells. NK cell activation was evaluated by surface staining of CD107a and was increased in the condition where the VSV coding for the TAME-IT molecule was used.

FIG. 8: KN41 induces greater activation of lymphocytes T. (A) Total PBMCs were co-cultured with M113 or M113-PD-L1 cells (10,000 tumor cells per well) at the indicated target:effector ratios in the presence or absence of beads coupled to anti-CD3/CD28 antibodies at a ratio one ball for 20 PBMC. The cells were either treated simultaneously with KN41 or pre-incubated with KN41 (KN41 PI). After 24 h, the supernatants were harvested and assayed for IFNg by ELISA. (B) Under the conditions with 5 PBMC per tumor cell and anti-CD3/CD28 beads, the IFNg in the supernatant was assayed at the indicated times. (C) Total CD8 LT sorted and previously amplified on feeder cells were co-cultured with M113 or M113-PD-L1 cells with two effector cells for a tumor cell in the presence of anti-CD3.CD28 beads (1:20). The cultures were treated or not with KN41 or an anti-PD-1 antibody (Nivolumab). IFNg in the supernatant was assayed at the indicated times

### EXAMPLES

### Example 1

### Aim

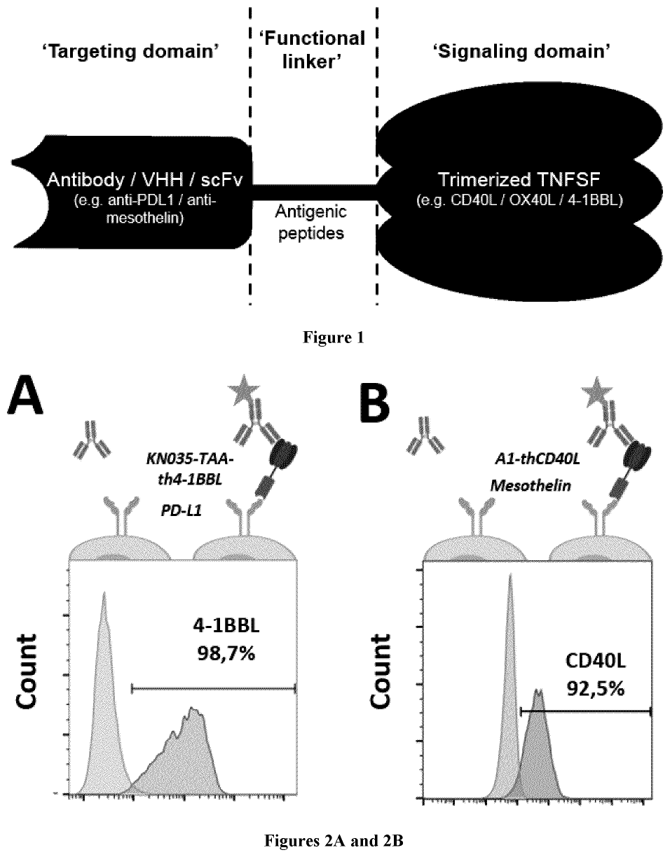

Demonstrate that the TAME-IT chimeric proteins (FIG. 1) can recognize specifically the cells of interest through binding of their ‘targeting domain’ to the targeted receptors.

### Material & Methods

### Protein Production

LentiX cells (HEK-293T) were plated at 7.10⁵ cells per well in a 6-well plate at day 0. At day 1, the medium was replaced by 2 mL of fresh DMEM complemented with 10%FCS. The cells were transfected using Lipofectamine 3000 with 2 μg of pcDNA3.1 plasmid coding for the TAME-IT proteins. 48 h post-transfection, the supernatants were collected and cleared by centrifugation at 10,000 g for 10 minutes.

Cell Staining 2.10⁵ Meso13 human mesothelioma cells or HEK-293T cells were incubated with the supernatants from control (EFGP) or TAME-IT-transfected HEK cells for 30 minutes. After 2 PBS washes, cells were stained with anti-h4-1BBL or anti-hCD40L antibodies (Biolegend, cat #311504 and 310806, respectively) for 15 minutes. After 2 PBS washes, cells were analyzed using a BD Accuri C1 flow cytometer.

### Results

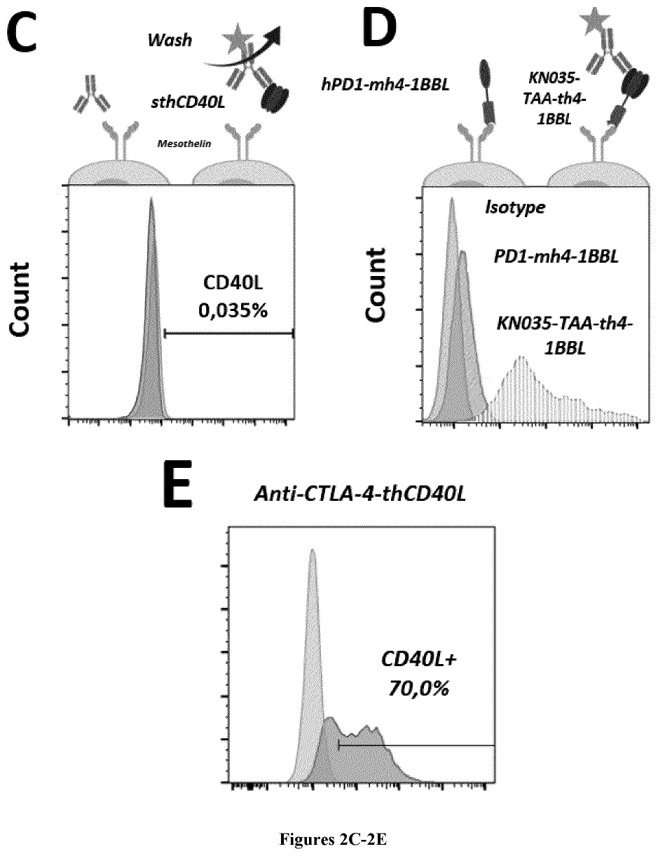

This experiment shows that the TAME-IT molecules that contain nanobodies directed against either PD-L1 (FIG. 2A) or Mesothelin (FIG. 2B) can recognize PD-L1⁺ and Mesothelin⁺ cells, respectively. The targeted cells are consequently neo-coated with the ‘signaling domains’ contained in the TAME-IT molecules, here trimerized 4-1BBL or CD40L. In additional experiments, we demonstrated that this fixation of the ‘targeting domains’ competes with other antibodies recognizing the same surface proteins, thus showing that the TAME-IT molecules are specific for their targets (data not shown). As shown in FIG. 2C, the presence of the targeting domain is absolutely necessary for this neo-coating as molecules lacking it will not recognize targeted cells. In FIG. 2D, we show that the TAME-IT constructions are better than competing molecules that use a human PD-1 extracellular domain in place of an anti-PD-L1 nanobody. We also showed that this specific targeting can be achieved by using as a ‘targeting domain’ a scFv against CTLA-4 (FIG. 2E), thus confirming that this domain can be made from different types of targeting molecules (e.g. nanobodies, scFv).

### Example 2

### Aim

Demonstrate that the membrane-bound TAME-IT proteins retain their ability to signal to facing immune cells through their ‘signaling domains’.

### Material & Methods

### Protein Production

LentiX cells (HEK-293T) were plated at 7.10⁵ cells per well in a 6-well plate at day 0. At day 1, the medium was replaced by 2 mL of fresh DMEM complemented with 10%FCS. The cells were transfected using lipofectamine 3000 with 2 μg of pcDNA3.1 plasmid coding for our constructions. 48 h post-transfection, the supernatants were collected and cleared by centrifugation at 10,000 g for 10 minutes.

### Dendritic Cell (DC) Differentiation

Human monocytes from healthy donors were plated at 2.10⁶ cell per mL and 1.10⁶ cells per cm² in 6-well plates in RPMI-1640 medium supplemented with 2%human albumin, granulocyte-macrophage colony-stimulating factor (GM-CSF, 500 IU/mL) and Interleukin-4 (IL-4, 50 ng/mL). After 5 days of culture (37° C., 5% CO2), the differentiated DCs were collected and counted.

### DC Maturation Assay

100,000 differentiated DCs were incubated for 48 h in a 96-well plate, either directly with the supernatants from HEK cells containing our molecules, or with tumor cells previously stained with these supernatants and washed before the co-culture.

### Cell Staining

Monocyte-derived DCs were collected by flushing at the end of the incubation time. After 2 PBS washes, they were stained with anti-hCD80, anti-hCD83, anti-hCD86 and anti-hHLA-DR antibodies for 30 minutes. After 2 PBS washes, cells were analyzed using a BD FACS Canto II flow cytometer. CD80, CD83 and CD86 expression was determined on the HLA-DR+ cell fraction corresponding to DCs.

### Results

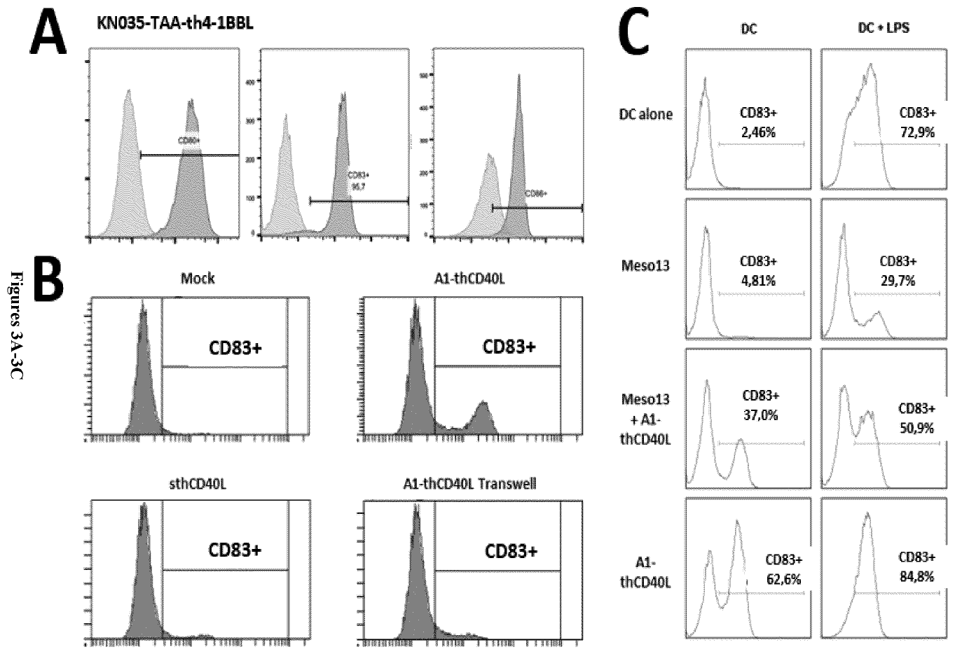

This experiment shows that the TAME-IT molecules can activate the maturation of human DCs, either in a soluble (FIG. 3A) or cell-bound (FIG. 3B) form, and when containing either a trimeric h4-1BBL (FIG. 3A) or a trimeric hCD40L (FIG. 3B-3C) domain. TAME-IT-induced DC maturation occurs despite the tumor cells (Meso13) being highly immunosuppressive (FIG. 3C). Synergism with pathogen-associated molecular patterns (PAMPs) is demonstrated as observed when co-treating DCs with both TAME-IT and LPS (FIG. 3C). Overall, it demonstrates that the signaling domains are fully functional and can readily activate human myeloid cells.

### Example 3

### Aim

Demonstrate that the TAME-IT molecules can be vectorized by being encoded in an oncolytic virus (OV) and efficiently produced by OV-infected cells.

### Material & Methods

### Virus Generation

The genes encoding for the TAME-IT proteins were inserted into the Vesicular Stomatitis Virus (VSV) Indiana strain genome by reverse genetics between VSV-G and VSV-L reading frames. The viruses were rescued in BHK-21 cells infected by MVA-T7 for 1 hour and then transfected with VSV-N, VSV-P, VSV-L-coding vectors and the anti-genomic VSV vector containing the TAME-IT sequence. Recombinant VSV were recovered from the supernatants by 0.22 μm filtration and several additional passages on BHK-21 cells. Similarly, the genes encoding for the TAME-IT proteins were inserted in the genome of an oncolytic strain of Vaccinia Virus by homologous recombination.

Cell infection

HEK-293T cells were plated at 7.10⁵ cells per well in 6-well plates and infected the day after at MOI-0.1 in 2 mL for overnight infection.

Supernatants were then collected and centrifuged at 10,000 g for 30 minutes. 1.5 mL of the supernatants were further concentrated 10 times on Vivaspin 5,000 Da. Twenty μL were then mixed with 5X laemmli/β-mercaptoethanol and heated at 95° C. for 5 minutes. Samples were loaded on a polyacrylamide gel and migrated at 200V for 40 minutes. Proteins were then transferred onto a PVDF membrane for 1 h at 100V. The membrane was blocked for 1 h in TBS/BSA and the proteins were subsequently detected with Strep-Tactin XT-HRP in TBS/BSA. Finally, the membrane was imaged by chemiluminescence using the Clarity™ Western ECL Substrate and a BioRad Chemidoc Imager.

### Results

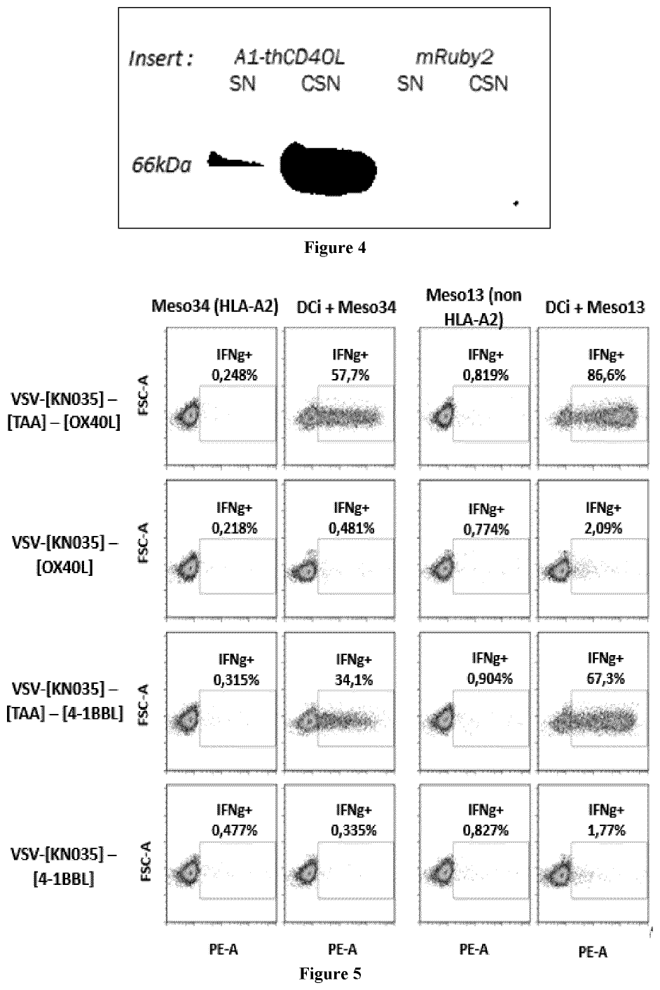

This experiment shows that the sequences of the TAME-IT proteins can be inserted in the genome of an oncolytic VSV and efficiently produced by VSV-infected tumor cells (FIG. 4). In parallel experiments, similar results were obtained when using an oncolytic VV coding for the TAME-IT proteins.

### Example 4

### Aim

Demonstrate the role of the ‘functional linker’ by determining whether the antigens encoded it contains can be processed and presented to CD4 and CD8 T cells.

### Material & Methods

Cell infection

Meso34 and Meso13 human mesothelioma cells were plated at 10⁴ cells per well in a 96-well plate at day 0 and infected at day 1 at MOI=0.1 with VSV encoding the different TAME-IT proteins. At day 2, 2.10⁵ DCs were added in the co-culture depending on the condition tested. At day 3, the DCs were flushed and plated in a V-bottom 96-well plate with 8.10⁵ T cell clones (CD4 or CD8 specific for the NY-ESO-1 antigen) for 6 h in presence or Brefeldin-A.

### Cell Staining

Cells were collected by flushing at the end of the incubation time. After 2 PBS washes, they were stained with an anti-hCD3 antibody for 30 minutes. After 2 PBS washes, cells were fixed in 4% paraformaldehyde, permeabilized with PBS+0.1% saponine and stained with an anti-hIFNγ antibody for 30 minutes. After 2 PBS+0.1% saponine washes, cells were analyzed using a BD Canto II flow cytometer. T cells were gated as CD3+ cells and analyzed for intracellular IFNγ production.

### Results

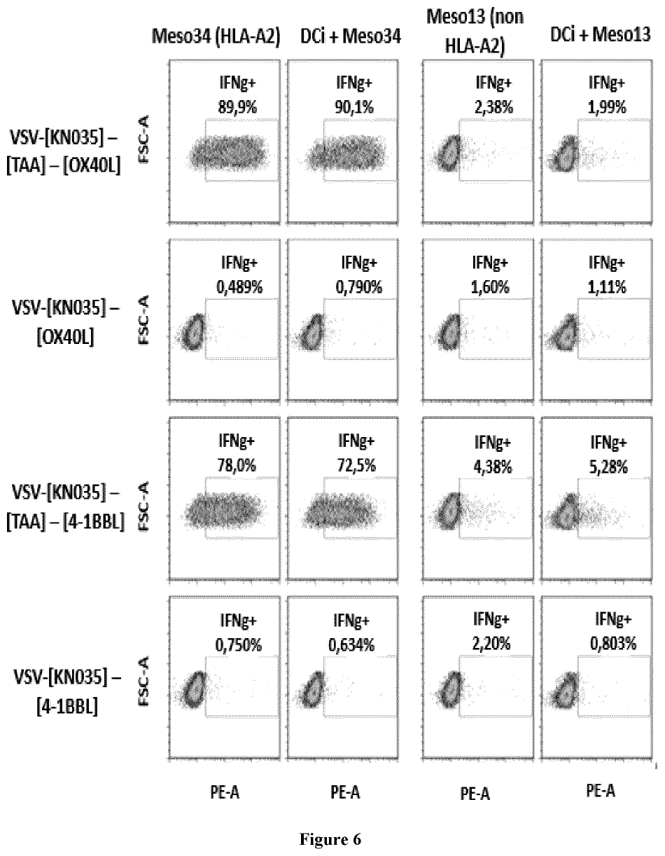

A first experiment using CD4 T cell clones shows that TAME-IT proteins containing a ‘functional linker’ are efficiently produced by tumor cells infected with a VSV encoding them (FIG. 5). These molecules are further processed and presented on MHC class II molecules by DCs having previously phagocytosed VSV-TAME-IT-infected tumor cells. These DCs subsequently activate TAA-specific CD4 T cells. This demonstrates that the ‘functional linker’ allows the specific activation of CD4 T cells that recognize antigenic peptides encoded in this linker. These results were confirmed in two additional experiments with DCs from different donors (data not shown).

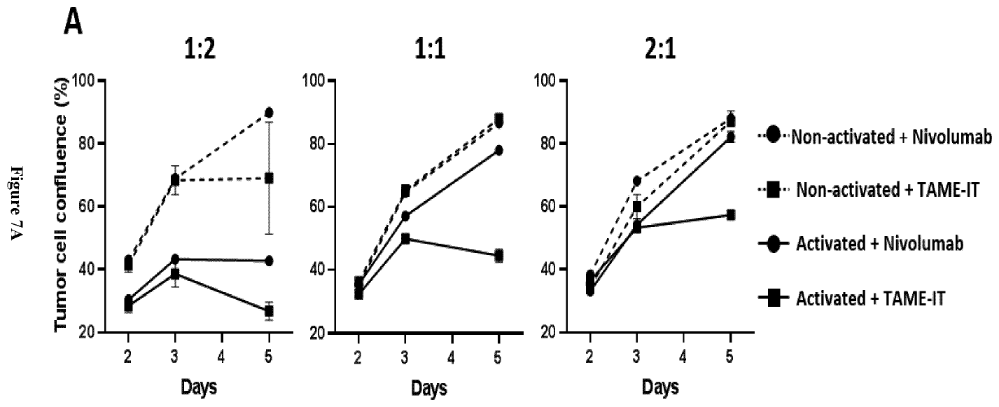

A second experiment shows that TAA peptides from the ‘functional linker’ are also efficiently processed and presented on MHC class I molecules to TAA-specific CD8 T cells by (i) tumor cells infected with VSV encoding for TAME-IT proteins and (ii) DCs having previously phagocytosed VSV-TAME-IT-infected tumor cells (FIG. 6). These results were confirmed in two additional experiments with DCs from different donors (data not shown).

### Example 5

### Aim

Demonstrate the activation of different types of human lymphocytes by the TAME-IT proteins.

### Material & Methods

### CD8 T Cell Activation

At day 0, PD-L1⁺ human melanoma cells were seeded in 96-well plate at a concentration of 10,000 cells per well. At day 1, human polyclonal CD8 T cells were added at ratios 2:1, 1:1, 1:2 onto tumor cells with or without activation beads previously coated with anti-CD3 and anti-CD28. In some conditions, the anti-PD1 antibody nivolumab or the purified TAME-IT protein KN035-TAA-th4-1BBL were added in the mix. The confluence of tumor cells was analyzed for 5 days by time-lapse microscopy.

NK cell activation

For NK activation analysis, 10⁴ MDA-MB-231 cells were plated in 96-well plate at day 0. At day 1, the cells were infected by VSV-KN035-TAA-th4-1BBL at MOI-0.1 for 18 h before a 6 h incubation with 10⁵ expanded human NK cells in presence of Brefeldin-A. NK cells were collected by flushing at the end of the incubation time. After 2 PBS washes, they were stained with an anti-hCD107a antibody for 30 minutes. After 2 PBS washes, cells were analyzed using a BD Accuri C1 flow cytometer.

### Results

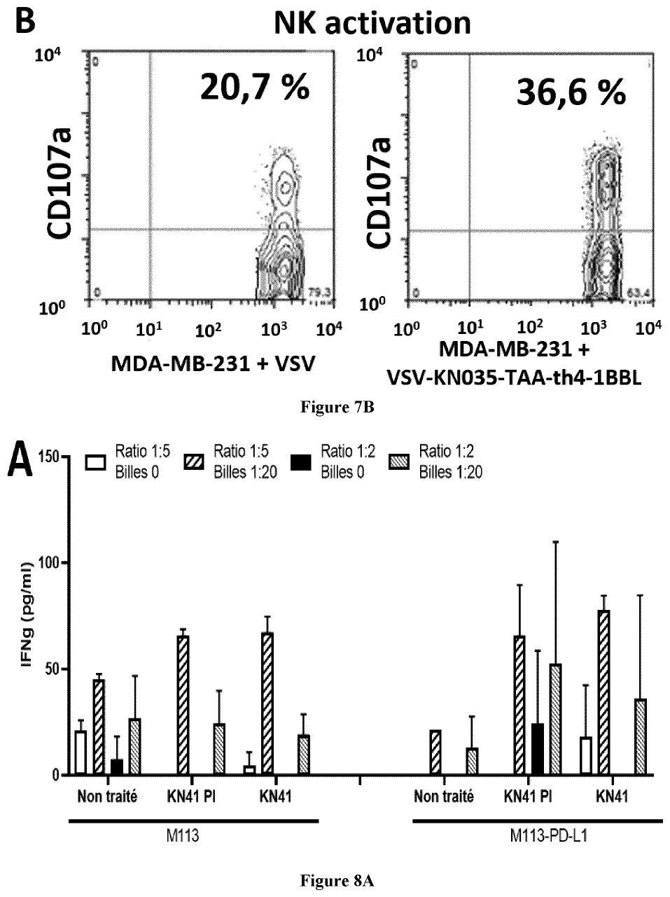

PD-L1⁺ human melanoma cells were co-cultured with activated polyclonal CD8 T cells in presence or not of the TAME-IT protein KN035-TAA-th4-1BBL (FIG. 7A). This experiment shows that the presence of the TAME-IT protein increased the killing of melanoma cells by CD8 T cells, which demonstrates its immunoactivating properties on T lymphocytes. In a similar way, when an oncolytic VSV coding for this TAME-IT protein was used to infect human breast cancer cells, co-cultured natural killer cells showed enhanced activation compared to control conditions (FIG. 7B).

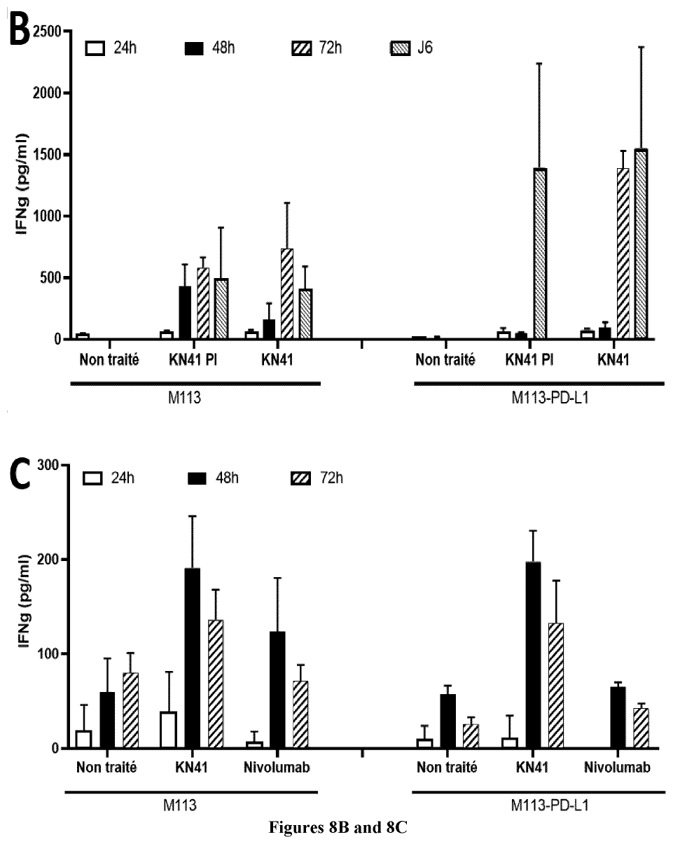

Moreover, KN41 induces greater activation of lymphocytes T (FIGS. 8A to 8C). These different results show that the TAME-IT proteins have the property to activate different types of human T lymphocytes in the context of anti-tumour immunity.

### REFERENCES

Throughout this application, various references describe the state of the art to which this invention pertains. The disclosures of these references are hereby incorporated by reference into the present disclosure.

1. A nucleic acid encoding a chimeric protein having a general structure of:
N terminus-(a)-(b)-(c)-C terminus, wherein: (a) is a signaling and/or targeting domain, (b) is a functional linker and (c) is a signaling and/or targeting domain or
N terminus-(c)-(b)-(a)-C terminus, wherein: (c) is a signaling and/or targeting domain, (b) is a functional linker and (a) is a signaling and/or targeting domain.

2. A chimeric protein having a general structure of:
N terminus-(a)-(b)-(c)-C terminus, wherein: (a) is a signaling and/or targeting domain, (b) is a functional linker and (c) is a signaling and/or targeting domain or
N terminus-(c)-(b)-(a)-C terminus, wherein: (c) is a signaling and/or targeting domain, (b) is a functional linker and (a) is a signaling and/or targeting domain.

3. The chimeric protein according to claim 2 wherein the signaling and/or targeting domain (a) is monomeric, homodimeric, heterodimeric, homotrimeric or heterotrimeric.

4. The chimeric protein according to claim 2 wherein the signaling and/or targeting domain (c) is monomeric, homodimeric, heterodimeric, homotrimeric or heterotrimeric.

5. The chimeric protein according to claim 2 wherein the signaling and/or targeting domain (a) is selected from anti-PD-L1, anti-CTLA-4, anti-TIGIT, anti-TIM-3, anti-VISTA, anti-BTLA, anti-LAG3, anti-CD28, anti-mesothelin (anti-MSLN), anti-CD19, and anti-HER2.

6. The chimeric protein according to claim 2 wherein the (b) functional linker is a tumor-associated antigens (TAA) or a tolerance antigens.

7. The chimeric protein according to claim 6 wherein the (TAA) is NY-ESO-1, MelanA antigen or MUC-1.

8. The chimeric protein according to claim 2 wherein the signaling and/or targeting domain (c) is a signaling domain selected from 4-1 BBL, OX-40 ligand (OX-40L), LIGHT (CD258), GITR ligand (GITRL), CD70, CD30 ligand, CD40 ligand (CD40L), TRAIL, and TL1A.

9. The chimeric protein according to claim 2 wherein the chimeric protein is [Anti-PD-L1]-[TAA]ₙ-[trimeric 4-1BBL], wherein n is equal to 1, 2, 3, 4, 5, 6, 7, 8, 9 or 10.

10. The chimeric protein according to claim 2 wherein the chimeric protein is [Anti-PD-L1]-[TAA]ₙ-[trimeric OX-40L], wherein n is equal to 1, 2, 3, 4, 5, 6, 7, 8, 9 or 10.

11. The chimeric protein according to claim 2 wherein the chimeric protein is [Anti-PD-L1]-[TAA]ₙ-[dimeric 4-1 BBL and monomeric OX-40L], wherein n is equal to 1, 2, 3, 4, 5, 6, 7, 8, 9 or 10.

12. A method of treating cancer or an inflammatory disease in a subject in need thereof comprising administering a therapeutically effective amount of a chimeric protein having a general structure of:
N terminus-(a)-(b)-(c)-C terminus, wherein: (a) is a signaling and/or targeting domain, (b) is a functional linker and (c) is a signaling and/or targeting domain or
N terminus-(c)-(b)-(a)-C terminus, wherein: (c) is a signaling and/or targeting domain, (b) is a functional linker and (a) is a signaling and/or targeting domain.

13. The method according to claim 12 wherein the inflammatory diseases is acne vulgaris, acute inflammation, allergic rhinitis, asthma, atherosclerosis, atopic dermatitis, autoimmune disease, autoinflammatory diseases, autosomal recessive spastic ataxia, bronchiectasis, celiac disease, chronic cholecystitis, chronic inflammation, chronic prostatitis, colitis, diverticulitis, familial eosinophilia (fe), glomerulonephritis, glycerol kinase deficiency, hidradenitis suppurativa, hypersensitivities, inflammation, inflammatory bowel diseases, inflammatory pelvic disease, interstitial cystitis, laryngeal inflammatory disease, Leigh syndrome, lichen planus, mast cell activation syndrome, mastocytosis, ocular inflammatory disease, otitis, pain, pelvic inflammatory disease, reperfusion injury, respiratory disease, restenosis, rheumatic fever, rheumatoid arthritis, rhinitis, sarcoidosis, septic shock, silicosis or other pneumoconioses, transplant rejection, tuberculosis, or vasculitis.

14. The method according to claim 13 wherein the autoimmune disease is multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, multiple sclerosis, myasthenia gravis, Reiter's syndrome, or Grave's disease.

15. An expression vector comprising the nucleic acid encoding the chimeric protein of claim 1.

16. The expression vector of claim 15 wherein the vector is a vesicular stomatitis virus (VSV), a lentivirus (LV), aretrovirus (RV), an adenovirus (AV), a measles virus, a herpesvirus, a vaccinia virus (VV), a myxoma virus, a reovirus, a parvovirus, a mumps virus or an adeno-associated viruses (AAV.
